(19) United States
(12) Patent Application Publication
(10) Pub. No.: US 2021/0101801 A1
(43) Pub. Date: Apr. 8, 2021

(54) A PROCESS FOR PREPARING A POROUS OXIDIC MATERIAL WHICH COMPRISES MICROPORES AND MESOPORES AND WHICH COMPRISES A ZEOLITIC MATERIAL HAVING A FRAMEWORK TYPE AEI

(71) Applicant: BASF SE, Ludwigshafen am Rhein (DE)

(72) Inventors: Robert MCGUIRE, Florham Park, NJ (US); Ulrich MUELLER, Neustadt (DE); Feng-Shou XIAO, Hangzhou (CN); Xiangju MENG, Hangzhou (CN); Xinhe BAO, Dalian City (CN); Xiulian PAN, Dalian City (CN); Toshiyuki YOKOI, Midori Ku (JP); Dirk DE VOS, Leuven (BE); Ute KOLB, Mainz (DE); Hermann GIES, Bochum (DE); Bernd MARLER, Bochum (DE); Weiping ZHANG, Dalian City (CN)

(73) Assignee: BASF SE, Ludwigshafen am Rhein (DE)

(21) Appl. No.: 17/045,624

(22) PCT Filed: Jan. 22, 2019
(86) PCT No.: PCT/CN2019/072638

§ 371 Date: Oct. 6, 2020

(30) Foreign Application Priority Data
Apr. 20, 2018 (CN) PCT/CN2018/083928

(51) Int. Cl. C01B 39/48; B01J 29/76; C01B 39/02; C07C 1/24

(52) U.S. Cl. CPC C01B 39/48; B01J 29/76; C01B 39/026; C07C 1/24; C07C 2529/76; C01P 2006/14; C01P 2006/12; C01P 2002/72; C01P 2006/16

(57) ABSTRACT

A process for preparing a porous oxidic material with micropores and mesopores and a zeolitic material having an AEI framework with a tetravalent element Y, a trivalent element X and oxygen, the micropores having a pore diameter determined by nitrogen adsorption-desorption at 77 K of less than 2 nm and the mesopores having a pore diameter of from 2 to 50 nm, the process involving subjecting a synthesis mixture to hydrothermal crystallization at a crystallization temperature of from 90 to 200° C., to obtain a mother liquor containing the porous oxidic material having the zeolitic AEI framework. The synthesis mixture may have a zeolitic material with an FAU framework comprising Y, X, and O, water, a base source, a first organic structure directing agent as an AEI framework type structure directing agent, a second organic structure directing agent with a dimethyl-octadecyl[3-(trimethoxysilyl)-propyl]ammonium cation, and seed crystals

The present invention relates to a process for preparing a porous oxidic material which comprises micropores and mesopores and which comprises a zeolitic material having a framework type AEI and having a framework structure comprising a tetravalent element Y, a trivalent element X and oxygen. Further, the present invention relates to a porous oxidic material which comprises micropores and mesopores and which comprises a zeolitic material having a framework type AEI and having a framework structure comprising a tetravalent element Y, a trivalent element X, and oxygen, and further relates to the use of said porous oxidic material as a catalytically active material, as a catalyst, or as a catalyst component.

Zeolitic materials having a framework type AEI are known to be potentially effective as catalysts or catalyst components in industrial applications, for example for converting nitrogen oxides (NOx) in an exhaust gas stream and for converting methanol-to-olefin (MTO). Synthetic AEI zeolitic materials may generally be produced by using organic templates. CN107285334 discloses a process for preparing a zeolitic material having a framework structure AEI using an alkyl piperidinium template and CN107285333 discloses a process for preparing a nano-sized zeolitic material having a framework type AEI using an alkyl piperidinium template and microwave heating. However, these processes do not permit to obtain a mesoporous zeolitic material having AEI framework type.

Therefore, it was an object of the present invention to provide a process for preparing a porous oxidic material which comprises micropores and mesopores and which comprises a zeolitic material having a framework type AEI having a framework structure comprising a tetravalent element Y, a trivalent element X and oxygen.

Surprisingly, it was found that the process according to the present invention permits to provide a porous oxidic material comprising a zeolitic material having a framework type AEI and both micropores and mesopores while being cost effective.

Therefore, the present invention relates to a process for preparing a porous oxidic material which comprises micropores and mesopores and which comprises a zeolitic material having a framework type AEI and having a framework structure comprising a tetravalent element Y, a trivalent element X and oxygen, wherein said micropores have a pore diameter determined according to nitrogen adsorption-desorption at 77 K of less than 2 nm and wherein said mesopores have a pore diameter determined according to nitrogen adsorption-desorption at 77 K in the range of from 2 to 50 nm, said process comprising:
(i) preparing a synthesis mixture comprising a zeolitic material having a framework type FAU and having a framework structure comprising the tetravalent element Y, the trivalent element X and oxygen, water, a source of base, a first organic structure directing agent being an AEI framework type structure directing agent, a second organic structure directing agent comprising a dimethyl-octadecyl[3-(trimethoxysilyl)propyl]ammonium cation containing compound, and seed crystals;
(ii) subjecting the mixture obtained in (i) to hydrothermal crystallization conditions comprising a crystallization temperature in the range of from 90 to 200° C., obtaining a mother liquor comprising the porous oxidic material comprising said zeolitic material having a framework type AEI;

wherein Y is one or more of Si, Sn, Ti, Zr and Ge;

wherein X is one or more of Al, B, In and Ga.

Preferably, the AEI framework type structure directing agent comprises a quaternary phosphonium cation containing compound, a N,N-diethyl-2,6-dimethylpiperidinium cation containing compound, a N,N-diethyl-3,5-dimethylpiperidinium cation containing compound, a N,N-dimethyl-2,6-dimethylpiperidinium cation containing compound, a N,N-dimethyl-3,5-dimethylpiperidinium cation containing compound, a N,N,N-trimethyl-1-adamantamonium cation containing compound, cis-2,6-dimethylpiperidinium cation containing compound, cis-trans-3,5-dimethylpiperidinium cation containing compound, a 2,2,7,7-tetramethyl-2-azabicyclo[4.1.1]octan-2-ium cation containing compound, a 1,3,3,6,6-pentamethyl-6-azabicyclo[3.2.1]octan-6-ium cation containing compound, or mixture thereof. More preferably, the AEI framework type structure directing agent comprises a quaternary phosphonium cation containing compound, a N,N-diethyl-2,6-dimethylpiperidinium cation containing compound. a N.N-diethyl-3,5-dimethylpiperidinium cation containing compound, a N,N,N-trimethyl-1-adamantamonium cation containing compound or mixture thereof. More preferably, the AEI framework type structure directing agent comprises a quaternary phosphonium cation containing compound, a N,N-diethyl-2,6-dimethylpiperidinium cation containing compound, a N,N-diethyl-3,5-dimethylpiperidinium cation containing compound or mixture thereof. More preferably, the AEI framework type structure directing agent comprises a quaternary phosphonium cation containing compound, a N,N-diethyl-2,6-dimethylpiperidinium cation containing compound or mixture thereof.

The quaternary phosphonium cation containing compound is preferably a R₁R₂R₃R₄P+-containing compound, wherein R₁, R₂, R₃, and R₄ independently from one another stand for optionally substituted and/or optionally branched (C₁-C₆)alkyl, more preferably (C₁-C₅)alkyl, more preferably (0₁-C₄)alkyl, more preferably (C₇-C₃)alkyl, and preferably for optionally substituted methyl or ethyl, more preferably R₁, R₂, R₃, and R₄ stand for optionally substituted ethyl, more preferably unsubstituted ethyl.

Preferably, the quaternary phosphonium cation containing compound is a salt, more preferably one or more of a hydroxide and a halide, more preferably one or more of an iodide, a chloride, a fluoride and a bromide, wherein more preferably the quaternary phosphonium cation containing compound comprises, more preferably is, a hydroxide.

The N,N-diethyl-2,6-dimethylpiperidinium cation containing compound is preferably one or more of a N,N-diethyl-trans-2,6-dimethylpiperidinium cation containing compound and a N.N-diethyl-cis-2.6-dimethylpiperidinium cation containing compound, more preferably a N,N-diethyl-cis-2,6-dimethylpiperidinium cation containing compound.

Preferably, the N,N-diethyl-2,6-dimethylpiperidinium cation containing compound is a salt, more preferably one or more of a hydroxide and a halide, more preferably one or more of an iodide, a chloride, a fluoride and a bromide, wherein more preferably the N,N-diethyl-2,6-dimethylpiperidinium cation containing compound comprises, more preferably is, a hydroxide.

The N,N-diethyl-3,5-dimethylpiperidinium cation containing compound is preferably one or more of a N,N-diethyl-trans-3,5-dimethylpiperidinium cation containing compound and a N,N-diethyl-cis-3,5-dimethylpiperidinium cation containing compound.

Preferably, the N,N-diethyl-3,5-dimethylpiperidinium cation containing compound is a salt, more preferably one or more of a hydroxide and a halide, more preferably one or more of an iodide, a chloride, a fluoride and a bromide, wherein more preferably the N,N-diethyl-3,5-dimethylpiperidinium cation containing compound comprises, more preferably is, a hydroxide.

The N.N-dimethyl-2,6-dimethylpiperidinium cation containing compound is preferably one or more of a N,N-dimethyl-trans-2,6-dimethylpiperidinium cation containing compound and a N,N-dimethyl-cis-2,6-dimethylpiperidinium cation containing compound.

Preferably, the N,N-dimethyl-2,6-dimethylpiperidinium cation containing compound is a salt, more preferably one or more of a hydroxide and a halide, more preferably one or more of an iodide, a chloride, a fluoride and a bromide, wherein more preferably the N,N-dimethyl-2,6-dimethylpiperidinium cation containing compound comprises, more preferably is, a hydroxide.

The N,N-dimethyl-3,5-dimethylpiperidinium cation containing compound is preferably one or more of a N,N-dimethyl-trans-3,5-dimethylpiperidinium cation containing compound and a N,N-dimethyl-cis-3,5-dimethylpiperidinium cation containing compound.

It is more preferred that the N,N-dimethyl-3,5-dimethylpiperidinium cation containing compound is a N,N-dimethyl-trans-3,5-dimethylpiperidinium cation containing compound or a N,N-dimethyl-cis-3,5-dimethylpiperidinium cation containing compound. Alternatively, it is more preferred that the N,N-dimethyl-3,5-dimethylpiperidinium cation containing compound is a mixture of a N,N-dimethyl-trans-3,5-dimethylpiperidinium cation containing compound and a N,N-dimethyl-cis-3,5-dimethylpiperidinium cation containing compound. It is more preferred that in the mixture of a N,N-dimethyl-trans-3,5-dimethylpiperidinium cation containing compound and a N,N-dimethyl-cis-3,5-dimethylpiperidinium cation containing compound, the ratio of the trans-isomer to the cis-isomer is of at least 15:85, more preferably of at least 20:80, more preferably of at least 30:70, more preferably of at least 60:40, more preferably in the range of from 60:40 to 85:15, more preferably in the range of from 50:50 to 80:20.

Preferably, the N,N-dimethyl-3,5-dimethylpiperidinium cation containing compound is a salt, more preferably one or more of a hydroxide and a halide, more preferably one or more of an iodide, a chloride, a fluoride and a bromide, wherein more preferably the N,N-dimethyl-3,5-dimethylpiperidinium cation containing compound comprises, more preferably is, a hydroxide.

The cis-2,6-dimethylpiperidinium cation containing compound is preferably a salt, more preferably one or more of a hydroxide and a halide, more preferably one or more of an iodide, a chloride, a fluoride and a bromide, wherein more preferably the cis-2,6-dimethylpiperidinium cation containing compound comprises, more preferably is, a hydroxide.

The cis-trans-3,5-dimethylpiperidinium cation containing compound is preferably a salt, more preferably one or more of a hydroxide and a halide, more preferably one or more of an iodide, a chloride, a fluoride and a bromide, wherein more preferably the cis-trans-3,5-dimethylpiperidinium cation containing compound comprises, more preferably is, a hydroxide.

The 2,2,7,7-tetramethyl-2-azabicyclo[4.1.1]octan-2-ium cation containing compound is preferably a salt, more preferably one or more of a hydroxide and a halide, more preferably one or more of an iodide, a chloride, a fluoride and a bromide.

The 1,3,3,6,6-pentamethyl-6-azabicyclo[3.2.1]octan-6-ium cation containing compound is preferably a salt, more preferably one or more of a hydroxide and a halide, more preferably one or more of an iodide, a chloride, a fluoride and a bromide.

More preferably, the AEI framework type structure directing agent comprises a N,N-diethyl-2,6-dimethylpiperidinium cation containing compound, wherein the N,N-diethyl-2,6-dimethylpiperidinium cation containing compound is one or more of a N,N-diethyl-trans-2,6-dimethylpiperidinium cation containing compound and a N,N-diethyl-cis-2,6-dimethylpiperidinium cation containing compound, more preferably a N,N-diethy-cis-2,6-dimethylpiperidinium cation containing compound.

It is preferred that the dimethyloctadecyl[3-(tri-methoxysilyl)propyl]ammonium cation containing compound is a salt, more preferably one or more of a hydroxide and a halide, more preferably a halide, more preferably one or more of an iodide, a chloride, a fluoride and a bromide, wherein more preferably the dimethyloctadecyl[3-(trimethoxysilyl)propyl]ammonium cation containing compound comprises, more preferably is, a chloride.

Preferably, from 92 to 100 weight-%, more preferably from 95 to 100 weight-%, more preferably from 98 to 100 weight-%, more preferably from 99 to 100 weight-%, more preferably from 99.5 to 100 weight-%, more preferably from 99.9 to 100 weight-%, of the porous oxidic material consist of the zeolitic material having a framework type AEI.

In the context of the present invention, impurities may be present in the porous oxidic material. Such impurities may be one or more zeolitic materials having a framework structure different than AEI. For example, such impurities may be one or more of a zeolitic material having a framework type MOR and a zeolitic material having a framework type FAU.

Preferably, the porous oxidic material consists of micropores, mesopores and the zeolitic material having a framework type AEI.

It is preferred that Y is Si.

Preferably, X is one or more of Al and B, more preferably Al. More preferably, Y is Si and X is Al.

It is preferred that the zeolitic material provided in (i) and having a framework type FAU is a zeolitic material selected from the group consisting of a faujasite zeolite, a zeolite Y, a zeolite X, an LSZ-210 zeolite, a zeolite US Y, and a mixture of two or more thereof, more preferably selected from the group consisting of zeolite Y, US Y and a mixture thereof. It is more preferred that the zeolitic material provided in (i) and having a framework type FAU is a zeolite US Y. Alternatively, it is more preferred that the zeolitic material provided in (i) and having a framework type FAU is a zeolite Y.

In the framework structure of the zeolitic material having a framework type FAU provided in (i), the molar ratio of Y:X, calculated as YO₂:X₂O₃, is preferably in the range of from 5:1 to 100:1, more preferably in the range of from 10:1 to 60:1, more preferably in the range of from 18:1 to 45:1, more preferably in the range of from 20:1 to 37:1, more preferably in the range of from 20:1 to 30:1.

It is more preferred that the zeolitic material provided in (1) and having a framework type FAU is a zeolite US Y, wherein in the framework structure of the zeolitic material having a framework type FAU provided in (i), the molar ratio of Y:X, calculated as YO₂:X₂O₃, is in the range of from 20:1 to 30:1. It is more preferred that the zeolitic material provided in (i) and having a framework type FAU is a zeolite Y, wherein in the framework structure of the zeolitic material having a framework type FAU provided in (i), the molar ratio of Y:X, calculated as YO₂:X₂O₃, is in the range of from 20:1 to 37:1.

In the synthesis mixture in (i), it is preferred that the molar ratio of the first organic structure directing agent, FOSDA, relative to Y, calculated as FOSDA:YO₂, is in the range of from 0.05:1 to 0.30:1, more preferably in the range of from 0.10:1 to 0.20:1.

In the synthesis mixture in (i), it is preferred that the molar ratio of the second organic structure directing agent, SOSDA, relative to Y, calculated as SOSDA:YO₂, is in the range of from 0.001:1 to 0.070:1, more preferably in the range of from 0.002:1 to 0.060:1. More preferably, in the synthesis mixture in (i), the molar ratio of the second organic structure directing agent, SOSDA, relative to Y, calculated as SOSDA:YO₂, is in the range of from 0.002:1 to 0.012:1, more preferably in the range of from 0.004:1 to 0.011:1, more preferably in the range of from 0.006:1 to 0.010:1, more preferably in the range of from 0.007:1 to 0.009:1. Alternatively, in the synthesis mixture in (i), the molar ratio of the second organic structure directing agent, SOSDA, relative to Y, calculated as SOSDA:YO₂, is more preferably in the range of from 0.006:1 to 0.022:1, more preferably in the range of from 0.010:1 to 0.020:1, more preferably in the range of from 0.013:1 to 0.017:1, more preferably in the range of from 0.015:1 to 0.018:1. As a further alternative, in the synthesis mixture in (i), the molar ratio of the second organic structure directing agent, SOSDA, relative to Y, calculated as SOSDA:YO₂, is more preferably in the range of from 0.018:1 to 0.040:1, more preferably in the range of from 0.021:1 to 0.028:1, more preferably in the range of from 0.023:1 to 0.026:1.

In the synthesis mixture in (i), the molar ratio of the second organic structure directing agent, SOSDA, relative to Y, calculated as SOSDA:YO₂, is more preferably in the range of from 0.007:1 to 0.026:1 or is in the range of from 0.007:1 to 0.017:1.

Therefore, the present invention preferably relates to a process for preparing a porous oxidic material which comprises micropores and mesopores and which comprises a zeolitic material having a framework type AEI and having a framework structure comprising a tetravalent element Y, a trivalent element X and oxygen, wherein said micropores have a pore diameter determined according to nitrogen adsorption-desorption at 77 K of less than 2 nm and wherein said mesopores have a pore diameter determined according to nitrogen adsorption-desorption at 77 K in the range of from 2 to 50 nm, said process comprising:
(i) preparing a synthesis mixture comprising a zeolitic material having a framework type FAU and having a framework structure comprising the tetravalent element Y, the trivalent element X and oxygen, water, a source of base, a first organic structure directing agent being an AEI framework type structure directing agent, a second organic structure directing agent comprising a dimethyl-octadecyl[3-(trimethoxysilyl)propyl]ammonium cation containing compound, and seed crystals;
(ii) subjecting the mixture obtained in (i) to hydrothermal crystallization conditions comprising a crystallization temperature in the range of from 90 to 200° C., obtaining a mother liquor comprising the porous oxidic material comprising said zeolitic material having a framework type AEI;

wherein Y is one or more of Si, Sn, Ti, Zr and Ge;

wherein X is one or more of Al, B, In and Ga,

wherein the AEI framework type structure directing agent comprises a N,N-diethyl-2,6-dimethylpiperidinium cation containing compound, wherein the N,N-diethyl-2,6-dimethylpiperidinium cation containing compound is one or more of a N,N-diethyl-trans-2,6-dimethylpiperidinium cation containing compound and a N,N-diethyl-cis-2,6-dimethylpiperidinium cation containing compound, more preferably a N,N-diethyl-cis-2,6-dimethylpiperidinium cation containing compound,

wherein, in the synthesis mixture in (i), the molar ratio of the first organic structure directing agent, FOSDA, relative to Y, calculated as FOSDA:YO₂, is in the range of from 0.05:1 to 0.30:1, more preferably in the range of from 0.10:1 to 0.20:1,

wherein, in the synthesis mixture in (i), the molar ratio of the second organic structure directing agent, SOSDA, relative to Y, calculated as SOSDA:YO₂, is in the range of from 0.001:1 to 0.070:1, more preferably in the range of from 0.002:1 to 0.060:1.

In the context of the present invention, in the synthesis mixture in (i), it is preferred that the molar ratio of the source of a base relative to Y, calculated as a source of a base:YO₂, is in the range of from 0.10:1 to 0.70:1, more preferably in the range of from 0.20:1 to 0.60:1, more preferably in the range of from 0.30:1 to 0.55:1. It is more preferred that in the synthesis mixture in (i), the molar ratio of the source of a base relative to Y, calculated as a source of a base:YO₂, is in the range of from 0.40:1 to 0.50:1, more preferably in the range of from 0.43:1 to 0.48:1, more preferably in the range of from 0.44:1 to 0.47:1. Alternatively, it is more preferred that in the synthesis mixture in (i), the molar ratio of the source of a base relative to Y, calculated as a source of a base:YO₂, is in the range of from 0.30:1 to 0.38:1, more preferably in the range of from 0.32:1 to 0.36:1.

It is more preferred that in the synthesis mixture in (i) which comprises a zeolite US Y, the molar ratio of the source of a base relative to Y, calculated as a source of a base:YO₂, is in the range of from 0.39:1 to 0.50:1, more preferably in the range of from 0.40:1 to 0.48:1, more preferably in the range of from 0.44:1 to 0.47:1. Alternatively, it is more preferred that in the synthesis mixture in (i) which comprises a zeolite Y, the molar ratio of the source of a base relative to Y, calculated as a source of a base:YO₂, is in the range of from 0.30:1 to 0.38:1, more preferably in the range of from 0.32:1 to 0.36:1.

It is more preferred that in the synthesis mixture in (i), the molar ratio of the source of a base relative to Y, calculated as a source of a base:YO₂, is in the range of from 0.32:1 to 0.47:1.

Preferably, in the synthesis mixture in (i), the molar ratio of H₂O relative to Y, calculated as H₂O:YO₂, is in the range of from 2:1 to 80:1, more preferably in the range of from 10:1 to 60:1, more preferably in the range of from 25:1 to 50:1, more preferably in the range of from 28:1 to 47:1, more preferably in the range of from 30:1 to 45:1.

There is no particular restriction as to the source of a base provided (i) provided that it permits to obtain a porous oxidic material which comprises micropores and mesopores and which comprises a zeolitic material having a framework type AEI and having a framework structure comprising a tetravalent element Y, a trivalent element X and oxygen. It is preferred that the source of a base provided in (i) comprises, more preferably is, a hydroxide.

It is preferred that the source of a base provided in (i) comprises, more preferably is, one or more of an alkali metal hydroxide and an alkaline earth metal hydroxide, more preferably an alkali metal hydroxide, more preferably sodium hydroxide.

In the context of the present invention, there is no particular restriction as to the type of seed crystals provided in (i). Preferably, the seed crystals provided in (i) comprise, more preferably consist of, a zeolitic material having a framework type selected from the group consisting of AEI, CHA and RTH, more preferably a zeolitic material having a framework type selected from the group consisting of AEI and CHA, wherein more preferably the seed crystals provided in (i) comprise, more preferably consist of, a zeolitic material having a framework type AEI.

Preferably, in the synthesis mixture (i), the weight ratio of the seed crystals to the zeolitic material having a framework type FAU is in the range of from 0.001:1 to 0.1:1, more preferably in the range of from 0.005:1 to 0.05:1, more preferably in the range of from 0.01:1 to 0.04:1.

Therefore, the present invention preferably relates to a process for preparing a porous oxidic material which comprises micropores and mesopores and which comprises a zeolitic material having a framework type AEI and having a framework structure comprising a tetravalent element Y, a trivalent element X and oxygen, wherein said micropores have a pore diameter determined according to nitrogen adsorption-desorption at 77 K of less than 2 nm and wherein said mesopores have a pore diameter determined according to nitrogen adsorption-desorption at 77 K in the range of from 2 to 50 nm, said process comprising:
(i) preparing a synthesis mixture comprising a zeolitic material having a framework type FAU and having a framework structure comprising the tetravalent element Y, the trivalent element X and oxygen, water, a source of base, a first organic structure directing agent being an AEI framework type structure directing agent, a second organic structure directing agent comprising a dimethyl-octadecyl[3-(trimethoxysilyl)propyl]ammonium cation containing compound, and seed crystals;
(ii) subjecting the mixture obtained in (i) to hydrothermal crystallization conditions comprising a crystallization temperature in the range of from 90 to 200 CC, obtaining a mother liquor comprising the porous oxidic material comprising said zeolitic material having a framework type AEI;

wherein Y is one or more of Si, Sn, Ti, Zr and Ge;

wherein X is one or more of Al, B, In and Ga;

wherein the AEI framework type structure directing agent comprises a N,N-diethyl-2,6-dimethylpiperidinium cation containing compound, wherein the N,N-diethyl-2,6-dimethylpiperidinium cation containing compound is one or more of a N,N-diethyl-trans-2,6-dimethylpiperidinium cation containing compound and a N,N-diethyl-cis-2,6-dimethylpiperidinium cation containing compound, more preferably a N,N-diethyl-cis-2,6-dimethylpiperidinium cation containing compound,

wherein, in the synthesis mixture in (i), the molar ratio of the first organic structure directing agent, FOSDA, relative to Y, calculated as FOSDA:YO₂, is in the range of from 0.05:1 to 0.30:1, more preferably in the range of from 0.10:1 to 0.20:1,

wherein, in the synthesis mixture in (i), the molar ratio of the second organic structure directing agent, SOSDA, relative to Y, calculated as SOSDA:YO₂, is in the range of from 0.001:1 to 0.070:1, more preferably in the range of from 0.002:1 to 0.060:1,

wherein, in the synthesis mixture in (i), the molar ratio of the source of a base relative to Y, calculated as a source of a base:YO₂, is in the range of from 0.10:1 to 0.70:1, more preferably in the range of from 0.20:1 to 0.60:1, more preferably in the range of from 0.30:1 to 0.55:1, more preferably in the range of from 0.32:1 to 0.47:1,

wherein, in the synthesis mixture (i), the weight ratio of the seed crystals to the zeolitic material having a framework type FAU is in the range of from 0.001:1 to 0.1:1, more preferably in the range of from 0.005:1 to 0.05:1, more preferably in the range of from 0.01:1 to 0.04:1.

In the context of the present invention, it is preferred that from 95 to 100 weight-%, more preferably from 98 to 100 weight-%, more preferably from 99 to 100 weight-%, more preferably from 99.5 to 100 weight-% of the synthesis mixture consist of the zeolitic material having a framework type FAU and having a framework structure comprising the tetravalent element Y, the trivalent element X and oxygen, water, the source of base, the first organic structure directing agent comprising an AEI framework type structure directing agent, the second organic structure directing agent comprising a dimethyloctadecyl[3-(trimethoxysilyl)propyl]ammonium cation containing compound and the seed crystals.

According to the present invention, there is no specific restriction on how the synthesis mixture is prepared in (i). Preferably, preparing the synthesis mixture in (i) comprises
(i.1) preparing a first mixture comprising the zeolitic material having a framework type FAU and having a framework structure comprising the tetravalent element Y, the trivalent element X and oxygen, water, and the first organic structure directing agent comprising an AEI framework type structure directing agent;
(i.2) adding the source of base to the first mixture obtained in (i.1), obtaining a second mixture;
(i.3) adding the second organic structure directing agent comprising a dimethyloctadecyl[3-(trimethoxysilyl)propyl]ammonium cation containing compound to the second mixture obtained in (i.2), obtaining a third mixture;
(i.4) adding the seed crystals to the third mixture obtained in (i.3), obtaining the synthesis mixture.

Preferably, preparing the first mixture in (i.1) comprises adding the first organic structure directing agent dropwise to the zeolitic material.

Preferably, preparing the first mixture in (i.1) comprises agitating, more preferably mechanically agitating, more preferably stirring, the mixture. As to (i.1), agitating is preferably performed at a temperature of the mixture in the range of from 12 to 35° C., more preferably in the range of from 15 to 30 ⁰0. As to (i.1), agitating is preferably performed for a duration in the range of from 0.10 to 3 hours, more preferably in the range of from 0.25 to 2 hours, more preferably in the range of from 0.4 to 1.75 hours, more preferably in the range of from 0.5 to 1.5 hours.

Preferably, preparing the second mixture according to (i.2) comprises agitating, more preferably mechanically agitating, more preferably stirring, the mixture. As to (i.2), agitating is preferably performed at a temperature of the mixture in the range of from 12 to 35° C., more preferably in the range of from 15 to 30° C. As to (i.2), agitating is preferably performed for a duration in the range of from 0.10 to 3 hours, more preferably in the range of from 0.25 to 2 hours, more preferably in the range of from 0.4 to 1.75 hours, more preferably in the range of from 0.5 to 1.5 hours.

Preferably, preparing the third mixture according to (i.3) preferably comprises agitating, more preferably mechanically agitating, more preferably stirring, the mixture. As to (i.3), agitating is preferably performed at a temperature of the mixture in the range of from 12 to 35° C., more preferably in the range of from 15 to 30° C. As to (i.3), agitating is preferably performed for a duration in the range of from 0.25 to 10 hours. It is more preferred that agitating according to (i.3) is performed for a duration in the range of from 0.25 to 4 hours, more preferably in the range of from 0.5 to 3 hours, more preferably in the range of from 1 to 2 hours. Alternatively, it is more preferred that agitating according to (i.3) is performed for a duration in the range of from 1 to 8 hours, more preferably in the range of from 2 to 6 hours, more preferably in the range of from 3 to 5 hours.

Preferably, preparing the synthesis mixture according to (i.4) preferably comprises agitating, more preferably mechanically agitating, more preferably stirring, the mixture. As to (i.4), agitating is preferably performed at a temperature of the mixture in the range of from 12 to 35° C., more preferably in the range of from 15 to 30° C. As to (i.4), agitating is preferably performed for a duration in the range of from 5 to 50 minutes. It is more preferred that agitating according to (i.4) is performed for a duration in the range of from 10 to 30 minutes, more preferably in the range of from 15 to 25 minutes. Alternatively, it is more preferred that agitating according to (i.4) is performed for a duration in the range of from 5 to 13 minutes.

According to (ii), the hydrothermal crystallization conditions preferably comprise a crystallization duration in the range of from 0.75 to 20 days, more preferably in the range of from 0.9 to 15 days, more preferably in the range of from 1 to 12 days, more preferably in the range of from 2 to 10 days, more preferably in the range of from 2 to 8 days. It is more preferred that according to (ii) the hydrothermal crystallization conditions comprise a crystallization duration in the range of from 4 to 8 days. Alternatively, it is more preferred that according to (ii) the hydrothermal crystallization conditions comprise a crystallization duration in the range of from 2 to 3.5 days.

It is more preferred that, when the synthesis mixture prepared in (i) comprises a zeolite US Y, according to (ii) the hydrothermal crystallization conditions comprise a crystallization duration in the range of from 4 to 8 days. It is more preferred that, when the synthesis mixture prepared in (i) comprises a zeolite Y, according to (ii) the hydrothermal crystallization conditions comprise a crystallization duration in the range of from 2 to 3.5 days.

According to (ii), the hydrothermal crystallization conditions preferably comprise a crystallization temperature in the range of from 100 to 180° C., more preferably in the range of from 120 to 160 ⁰0, more preferably in the range of from 130 to 150° C.

It is preferred that, during the hydrothermal crystallization according to (ii), the mixture obtained in (i) and subjected to (ii) is agitated, more preferably mechanically agitated, more preferably stirred.

According to (ii), it is preferred that subjecting the synthesis mixture obtained in (i) to hydro-thermal crystallization conditions is carried out under autogenous pressure, more preferably in an autoclave.

According to the present invention, it is preferred that the process further comprises
(iii) cooling the mother liquor comprising the porous oxidic material comprising the zeolitic material having a framework type AEI obtained from (ii), more preferably to a temperature in the range of from 10 to 50° C.

It is preferred that the process according to the present invention further comprises
(iv) separating the porous oxidic material from the mother liquor obtained from (ii) or (iii), more preferably from (iii).

Preferably, (iv) comprises
(iv.1) subjecting the mother liquor obtained from (ii) or more preferably from (iii), to a solid-liquid separation method, more preferably comprising a filtration method;
(iv.2) more preferably washing the porous oxidic material obtained from (iv.1);
(iv.3) drying the porous oxidic material obtained from (iv.1) or (iv.2), more preferably from (iv.2).

As to (iv.2), the porous oxidic material is preferably washed with water, more preferably with deionized water.

As to (iv.3), the porous oxidic material is preferably dried in a gas atmosphere having a temperature in the range of from 60 to 200° C., more preferably in the range of from 80 to 140° C., more preferably in the range of from 90 to 110° C.

As to (iv.3), the porous oxidic material is preferably dried in a gas atmosphere for a duration in the range of from 0.5 to 5 hours, more preferably in the range of from 1 to 4 hours, more preferably in the range of from 1 to 3 hours,

More preferably, the gas atmosphere in (iv.3) comprises, more preferably is, one or more of air, lean air, and oxygen, more preferably air.

Therefore, the present invention preferably relates to a process for preparing a porous oxidic material which comprises micropores and mesopores and which comprises a zeolitic material having a framework type AEI and having a framework structure comprising a tetravalent element Y, a trivalent element X and oxygen, wherein said micropores have a pore diameter determined according to nitrogen adsorption-desorption at 77 K of less than 2 nm and wherein said mesopores have a pore diameter determined according to nitrogen adsorption-desorption at 77 K in the range of from 2 to 50 nm, said process comprising:
(i) preparing a synthesis mixture comprising a zeolitic material having a framework type FAU and having a framework structure comprising the tetravalent element Y, the trivalent element X and oxygen, water, a source of base, a first organic structure directing agent being an AEI framework type structure directing agent, a second organic structure directing agent comprising a dimethyl-octadecyl[3-(trimethoxysilyl)propyl]ammonium cation containing compound, and seed crystals;
(ii) subjecting the mixture obtained in (i) to hydrothermal crystallization conditions comprising a crystallization temperature in the range of from 90 to 200° C., obtaining a mother liquor comprising the porous oxidic material comprising said zeolitic material having a framework type AEI;
(iii) cooling the mother liquor comprising the porous oxidic material comprising the zeolitic material having a framework type AEI obtained from (ii), more preferably to a temperature in the range of from 10 to 50° C.;
(iv) separating the porous oxidic material from the mother liquor obtained from (ii) or (iii), more preferably from (iii);

wherein Y is one or more of Si, Sn, Ti, Zr and Ge;

wherein X is one or more of Al, B, In and Ga,

wherein the AEI framework type structure directing agent comprises a N,N-diethyl-2,6-dimethylpiperidinium cation containing compound, wherein the N,N-diethyl-2,6-dimethylpiperidinium cation containing compound is one or more of a N,N-diethyl-trans-2,6-dimethylpiperidinium cation containing compound and a N,N-diethyl-cis-2,6-dimethylpiperidinium cation containing compound, more preferably a N,N-diethyl-cis-2.6-dimethylpiperidinium cation containing compound,

wherein, in the synthesis mixture in (i), the molar ratio of the first organic structure directing agent, FOSDA, relative to Y, calculated as FOSDA:YO₂, is in the range of from 0.05:1 to 0.30:1, more preferably in the range of from 0.10:1 to 0.20:1,

wherein, in the synthesis mixture in (i), the molar ratio of the second organic structure directing agent, SOSDA, relative to Y, calculated as SOSDA:YO₂, is in the range of from 0.001:1 to 0.070:1, more preferably in the range of from 0.002:1 to 0.060:1,

wherein, in the synthesis mixture in (i), the molar ratio of the source of a base relative to Y, calculated as a source of a base:YO₂, is in the range of from 0.10:1 to 0.70:1, more preferably in the range of from 0.20:1 to 0.60:1, more preferably in the range of from 0.30:1 to 0.55:1, more preferably in the range of from 0.32:1 to 0.47:1,

wherein, in the synthesis mixture (i), the weight ratio of the seed crystals to the zeolitic material having a framework type FAU is in the range of from 0.001:1 to 0.1:1, more preferably in the range of from 0.005:1 to 0.05:1, more preferably in the range of from 0.01:1 to 0.04:1.

In the context of the present invention, it is preferred that the process further comprises
(v) calcining the porous oxidic material obtained from (iv), preferably from (iv.3), in a gas atmosphere. More preferably, the gas atmosphere is air.

As to (v), the porous oxidic material is preferably calcined in a gas atmosphere having a temperature in the range of from 300 to 550° C.

As to (v), it is preferred that the porous oxidic material obtained from calcination has a total organic carbon content of at most 0.1 weight-%.

According to the present invention, it is preferred that said micropores have a micropore volume and said mesopores have a mesopore volume and wherein the ratio of mesopore volume to micropore volume of the porous oxidic material is of at least 0.5:1 and the ratio of rnesopore volume to the total pore volume of the porous oxidic material is of at least 0.3:1.

Preferably, said micropores have a micropore volume and said mesopores have a rnesopore volume, wherein the ratio of mesopore volume to micropore volume of the porous oxidic material is in the range of from 0.5:1 to 3:1, more preferably in the range of from 0.6:1 to 2:1.

It is preferred that said micropores have a micropore volume and said mesopores have a mesopore volume, wherein the ratio of mesopore volume to micropore volume of the porous oxidic material is in the range of from 0.75:1 to 2.5:1, more preferably in the range of from 1:1 to 2.1:1, more preferably in the range of from 1.35:1 to 2:1. Alternatively, it is preferred that said micropores have a micropore volume and said mesopores have a mesopore volume, wherein the ratio of mesopore volume to micropore volume of the porous oxidic material is in the range of from 0.55:1 to 2:1, more preferably in the range of from 0.6:1 to 1.25:1.

It is preferred that said micropores have a micropore volume and said mesopores have a mesopore volume, wherein the ratio of mesopore volume to the total pore volume of the porous oxidic material is in the range of from 0.3:1 to 1:1, more preferably in the range of from 0.35:1 to 0.95:1, more preferably in the range of from 0.38:1 to 0.7:1.

It is preferred that said micropores have a micropore volume and said mesopores have a mesopore volume, wherein the ratio of mesopore volume to the total pore volume of the porous oxidic material is in the range of from 0.4:1 to 0.9:1, more preferably in the range of from 0.50:1 to 0.75:1, more preferably in the range of 0.55:1 to 0.7:1. Alternatively, it is preferred that said micropores have a micropore volume and said mesopores have a mesopore volume, wherein the ratio of mesopore volume to the total pore volume of the porous oxidic material is in the range of from 0.35:1 to 0.6:1, more preferably in the range of from 0.38:1 to 0.55:1.

In the context of the present invention, the terms “total pore volume of the porous oxidic material” and “total pore volume” refer to the sum of the mesopore volume of the porous oxidic material and the micropore volume of a porous oxidic material.

Preferably, said mesopores of the porous oxidic material have a mesopore volume, determined as described in Reference Example 1 b), in the range of from 0.15 to 0.80 cm³/g.

It is more preferred that the mesopores of the porous oxidic material have a mesopore volume, determined as described in Reference Example 1 b), in the range of from 0.20 to 0.65 cm³/g, more preferably in the range of from 0.25 to 0.55 cm³/g, more preferably in the range of from 0.30 to 0.50 cm³/g. More preferably, the mesopore volume, determined as described in Reference Example 1 b), is in the range of from 0.30 to 0.40 cm³/g, more preferably in the range of from 0.32 to 0.38 cm³/g. As an alternative, more preferably the mesopore volume, determined as described in Reference Example 1 b), is in the range of from 0.40 to 0.50 cm³/g, more preferably in the range of from 0.42 to 0.48 cm³/g.

Alternatively, it is more preferred that the mesopores of the porous oxidic material have a mesopore volume, determined as described in Reference Example 1 b), in the range of from 0.15 to 0.50 cm³/g, preferably in the range of from 0.15 to 0.40 cm³/g, more preferably in the range of from 0.16 to 0.30 cm³/g.

It is preferred that said micropores of the porous oxidic material have a micropore volume, determined as described in Reference Example 1 b), in the range of from 0.05 to 0.50 cm³/g, more preferably in the range of from 0.10 to 0.40 cm³/g, more preferably in the range of from 0.20 to 0.30 cm³/g.

According to the present invention, the process preferably further comprises
(vi) subjecting the porous oxidic material obtained from (iv) or (v), more preferably from (iv.3) or (v), to ion-exchange conditions.

In particular, (vi) preferably comprises
(vi.1) subjecting the porous oxidic material obtained from (iv) or (v), more preferably from (iv.3) or (v), to ion-exchange conditions comprising bringing a solution comprising ammonium ions in contact with the porous oxidic material obtained from (iv) or (v), obtaining a porous oxidic material in its ammonium form.

More preferably, the solution comprising ammonium ions according to (vi.1) is an aqueous solution comprising a dissolved ammonium salt, preferably a dissolved inorganic ammonium salt, more preferably a dissolved ammonium nitrate.

According to (vi.1), it is preferred that the solution comprising ammonium ions according to (vi.1) has an ammonium concentration in the range of from 0.10 to 3 mol/l, more preferably in the range of from 0.20 to 2 mol/l, more preferably in the range of from 0.5 to 1.5 mol/l.

According to (vi.1), it is preferred that the solution comprising ammonium ions is brought in contact with the zeolitic material obtained from (iv) or (v) at a temperature of the solution in the range of from 60 to 100° C., more preferably in the range of from 70 to 90° C.

According to (vi.1), it is preferred that the solution comprising ammonium ions is brought in contact with the zeolitic material obtained from (iv) or (v) for a period of time in the range of from 1 to 6 hours, more preferably in the range of from 1.5 to 4 hours.

According to (vi.1), bringing the solution in contact with the porous oxidic material preferably comprises one or more of impregnating the porous oxidic material with the solution and spraying the solution onto the porous oxidic material, more preferably impregnating the porous oxidic material with the solution.

Preferably, (vi) further comprises
(vi.2) calcining the porous oxidic material obtained in (vi.1) in a gas atmosphere, more preferably in a gas atmosphere having a temperature in the range of from 450 to 650° C., more preferably in the range of from 500 to 600° C., obtaining the H-form of the porous oxidic material.

According to (iv.2), calcining preferably is performed in gas atmosphere for a duration in the range of from 2 to 6 hours, more preferably in the range of from 3 to 5 hours.

Preferably, (vi.1) and (vi.2) are carried out at least once, more preferably twice.

Preferably, as to (vi.2), the gas atmosphere comprises, more preferably is, one or more of air, lean air, and oxygen, more preferably air.

Preferably, (vi) further comprises
(vi.3) subjecting the porous oxidic material obtained from (vi.2) to ion-exchange conditions comprising bringing a solution comprising ions of one or more transition metals, more preferably of one or more of Cu and Fe, more preferably Cu, in contact with the porous oxidic material obtained from (vi.2).

According to (vi.3), it is preferred that the solution comprising ions of one or more transition metals is an aqueous solution comprising a dissolved salt of one or more transition metals, more preferably a dissolved organic copper salt, more preferably a dissolved copper acetate.

According to (vi.3), it is preferred that the solution comprising ions of one or more transition metals has a transition metal concentration, more preferably a copper concentration, in the range of from 0.10 to 3 mol/l, more preferably in the range of from 0.20 to 2 moll!, more preferably in the range of from 0.5 to 1.5 mol/l.

According to (vi.3), the solution comprising ions of one or more transition metals is preferably brought in contact with the porous oxidic material obtained from (vi.2) at a temperature of the solution in the range of from 60 to 100° C., more preferably in the range of from 70 to 90° C.

According to (vi.3), the solution comprising ions of one or more transition metals is preferably brought in contact with the zeolitic material obtained from (vi.2) for a period of time in the range of from 0.5 to 3 hours, more preferably in the range of from 0.5 to 2 hours.

It is preferred that (vi) further comprises
(vi.4) calcining the porous oxidic material obtained in (vi.3) in a gas atmosphere, more preferably in a gas atmosphere having a temperature in the range of from 450 to 650° C., more preferably in the range of from 500 to 600° C.

As to (vi.4), calcining is preferably performed in gas atmosphere for a duration in the range of from 1 to 6 hours, more preferably in the range of from 3 to 5 hours.

As to (vi.4), the gas atmosphere preferably comprises, more preferably is, one or more of air, lean air, and oxygen, more preferably air.

Therefore, the present invention preferably relates to a process for preparing a porous oxidic material which comprises micropores and mesopores and which comprises a zeolitic material having a framework type AEI and having a framework structure comprising a tetravalent element Y, a trivalent element X and oxygen, wherein said micropores have a pore diameter determined according to nitrogen adsorption-desorption at 77 K of less than 2 nm and wherein said mesopores have a pore diameter determined according to nitrogen adsorption-desorption at 77 K in the range of from 2 to 50 nm, said process comprising:
(i) preparing a synthesis mixture comprising a zeolitic material having a framework type EAU and having a framework structure comprising the tetravalent element Y, the trivalent element X and oxygen, water, a source of base, a first organic structure directing agent being an AEI framework type structure directing agent, a second organic structure directing agent comprising a dimethyl-octadecyl[3-(trimethoxysilyl)propyl]ammonium cation containing compound, and seed crystals;
(ii) subjecting the mixture obtained in (i) to hydrothermal crystallization conditions comprising a crystallization temperature in the range of from 90 to 200° C., obtaining a mother liquor comprising the porous oxidic material comprising said zeolitic material having a framework type AEI;
(iii) cooling the mother liquor comprising the porous oxidic material comprising the zeolitic material having a framework type AEI obtained from (ii), more preferably to a temperature in the range of from 10 to 50° C.;
(iv) separating the porous oxidic material from the mother liquor obtained from (ii) or (iii), preferably from (iii);
(vi) subjecting the porous oxidic material obtained from (iv) or (v), more preferably from (iv.3) or (v), to ion-exchange conditions;

wherein Y is one or more of Si, Sn, Ti, Zr and Ge;

wherein X is one or more of Al, B, In and Ga,

wherein the AEI framework type structure directing agent comprises a N,N-diethyl-2.6-dimethylpiperidinium cation containing compound, wherein the N,N-diethyl-2,6-dimethylpiperidinium cation containing compound is one or more of a N,N-diethyl-trans-2,6-dimethylpiperidinium cation containing compound and a N,N-diethyl-cis-2,6-dimethylpiperidinium cation containing compound, more preferably a N,N-diethyl-cis-2,6-dimethylpiperidinium cation containing compound,

wherein, in the synthesis mixture in (i), the molar ratio of the first organic structure directing agent, FOSDA, relative to Y, calculated as FOSDA:YO₂, is in the range of from 0.05:1 to 0.30:1, more preferably in the range of from 0.10:1 to 0.20:1,

wherein, in the synthesis mixture in (i), the molar ratio of the second organic structure directing agent, SOSDA, relative to Y, calculated as SOSDA:YO₂, is in the range of from 0.001:1 to 0.070:1, more preferably in the range of from 0.002:1 to 0.060:1,

wherein, in the synthesis mixture in (i), the molar ratio of the source of a base relative to Y, calculated as a source of a base:YO₂, is in the range of from 0.10:1 to 0.70:1, more preferably in the range of from 0.20:1 to 0.60:1, more preferably in the range of from 0.30:1 to 0.55:1,

wherein, in the synthesis mixture (i), the weight ratio of the seed crystals to the zeolitic material having a framework type FAU is in the range of from 0.001:1 to 0.1:1, more preferably in the range of from 0.005:1 to 0.05:1, more preferably in the range of from 0.01:1 to 0.04:1.

In the context of the present invention, it is preferred that the process further comprises
(vii) ageing the porous oxidic material obtained in (vi.2), more preferably in (vi.4), in gas atmosphere.

Ageing in (vii) is preferably performed in gas atmosphere, more preferably in air, having a temperature in the range of from 400 to 1000° C., more preferably in the range of from 600 to 800° C. As to (vii), ageing is preferably performed for a duration in the range of from 5 to 100 hours, more preferably in the range of from 10 to 60 hours.

As to (vii), it is preferred that the gas atmosphere comprises, more preferably is, one or more of air, lean air, and oxygen, more preferably air.

It is preferred that the process of the present invention consists of (i) and (ii), more preferably of (i), (ii) and (iv), and more preferably (i), (ii), (iv) and (v). It is more preferred that the process of the present invention consists of (i), (ii), (iii), (iv) and (v), more preferably of (i), (ii), (iii), (iv), (v), and (vi). It may be more preferred that the process of the present invention consists of (i), (ii), (iii), (iv), (v), (vi) and (vii).

The present invention further relates to a porous oxidic material which comprises micropores and mesopores and which comprises a zeolitic material having a framework type AEI and having a framework structure comprising a tetravalent element Y, a trivalent element X, and oxygen, wherein said micropores have a pore diameter determined according to nitrogen adsorption-desorption at 77 K of less than 2 nm and wherein said mesopores have a pore diameter determined according to nitrogen adsorption-desorption at 77 K in the range of from 2 to 50 nm, wherein Y is one or more of Si, Sn, Ti, Zr, and Ge and wherein X is one or more of Al, B, In, and Ga. wherein said micropores have a micropore volume and said mesopores have a mesopore volume, the ratio of mesopore volume to micropore volume being of at least 0.5:1 and the ratio of mesopore volume to the total pore volume of the porous oxidic material being of at least 0.3:1, wherein the porous oxidic material is obtainable or obtained by a process according to the present invention.

The present invention further relates to a porous oxidic material which comprises micropores and mesopores and which comprises a zeolitic material having a framework type AEI and having a framework structure comprising a tetravalent element Y, a trivalent element X, and oxygen, wherein said micropores have a pore diameter determined according to nitrogen adsorption-desorption at 77 K of less than 2 rim arid wherein said mesopores have a pore diameter determined according to nitrogen adsorption-desorption at 77 K in the range of from 2 to 50 rim, wherein Y is one or more of Si, Sn, Ti, Zr, and Ge and wherein X is one or more of Al, B, In, and Ga, wherein said micropores have a micropore volume and said mesopores have a mesopore volume, the ratio of mesopore volume to micropore volume being of at least 0.5:1 and the ratio of mesopore volume to the total pore volume of the porous oxidic material being of at least 0.3:1, wherein the porous oxidic material is preferably obtainable or obtained by a process according to the present invention.

Preferably, Y is Si and X is one or more of Al and B. More preferably, Y is Si and X is Al.

It is preferred that the zeolitic material having a framework type AEI is a zeolite SSZ-39.

Preferably, from 92 to 100 weight-%, more preferably from 95 to 100 weight-%, more preferably from 98 to 100 weight-%, more preferably from 99 to 100 weight-%, more preferably from 99.5 to 100 weight-%, more preferably from 99.9 to 100 weight-%, of the porous oxidic material consist of a zeolitic material having a framework type AEI.

In the context of the present invention, impurities may be present in the porous oxidic material. Such impurities may be one or more zeolitic materials having a framework structure different than AEI. For example, such impurities may be one or more of a zeolitic material having a framework type MOR and a zeolitic material having a framework type FAU.

Preferably, in the framework structure of the zeolitic material having framework type AEI, the molar ratio of Y:X, calculated as a YO₂:X₂O₃, is in the range of from 2:1 to 40:1, more preferably in the range of from 10:1 to 30:1, more preferably in the range of from 14:1 to 26:1, more preferably in the range of from 16:1 to 24:1.

It is preferred that the porous oxidic material has a BET specific surface area, determined as described in Reference Example 1 b), in the range of from 500 to 900 m²/g, more preferably in the range of from 540 to 820 m²/g.

It is preferred that the porous oxidic material has a BET specific surface area, determined as described in Reference Example 1 b), in the range of from 600 to 900 m²/g, more preferably in the range of from 650 to 850 m²/g, more preferably in the range of from 750 to 830 m²/g, more preferably in the range of from 785 to 820 m²/g. Alternatively, it is preferred that the porous oxidic material preferably has a BET specific surface area, determined as described in Reference Example 1 b), in the range of from 520 to 600 m²/g, more preferably in the range of from 540 to 575 m²/g.

Preferably, the mesopore volume, determined as described in Reference Example 1 b), is in the range of from 0.15 to 0.80 cm³/g, more preferably in the range of from 0.15 to 0.50 cm³/g, more preferably in the range of from 0.16 to 0.48 cm³/g.

It is preferred that the mesopore volume, determined as described in Reference Example 1 b), is in the range of from 0.20 to 0.65 cm³/g, more preferably in the range of from 0.25 to 0.55 cm³/g, more preferably in the range of from 0.30 to 0.50 cm³/g. More preferably, the mesopore volume, determined as described in Reference Example 1 b), is in the range of from 0.30 to 0.40 cm³/g, more preferably in the range of from 0.32 to 0.38 cm³/g. Alternatively, more preferably the mesopore volume, determined as described in Reference Example 1 b), is in the range of from 0.40 to 0.50 cm³/g, more preferably in the range of from 0.42 to 0.48 cm³/g.

Alternatively, it is preferred that the mesopore volume, determined as described in Reference Example 1 b), is in the range of from 0.15 to 0.50 cm³/g, more preferably in the range of from 0.15 to 0.40 cm³/g, more preferably in the range of from 0.16 to 0.30 cm³/g.

It is preferred that the micropore volume, determined as described in Reference Example 1 b), is in the range of from 0.05 to 0.50 cm³/g, more preferably in the range of from 0.10 to 0.40 cm³/g, more preferably in the range of from 0.20 to 0.30 cm³/g.

Preferably, the ratio of mesopore volume to micropore volume is in the range of from 0.5:1 to 3:1. More preferably, the ratio of mesopore volume to micropore volume is in the range of from 0.75:1 to 2.5:1, more preferably in the range of from 1:1 to 2.1:1, more preferably in the range of from 1.35:1 to 2:1. Alternatively, it is more preferred that the ratio of mesopore volume to micropore volume is in the range of from 0.55:1 to 2:1, more preferably in the range of from 0.6:1 to 1.25:1.

Preferably, the ratio of mesopore volume to the total pore volume of the porous oxidic material is in the range of from 0.3:1 to 1:1, more preferably in the range of from 0.35:1 to 0.95:1, more preferably in the range of from 0.38:1 to 0.7:1.

It is preferred that the ratio of mesopore volume to the total pore volume is in the range of from 0.4:1 to 0.9:1, more preferably in the range of from 0.50:1 to 0.75:1, more preferably in the range of 0.55:1 to 0.7:1. Alternatively, it is preferred that the ratio of mesopore volume to the total pore volume is in the range of from 0.35:1 to 0.6:1, more preferably in the range of from 0.38:1 to 0.55:1.

It is preferred that the porous oxidic material has a crystallinity, determined as described in Reference Example 1 e), in the range of from 80 to 100%, preferably in the range of from 90 to 100%, more preferably in the range of from 99 to 100%.

It is preferred that the zeolitic material having a framework type AEI has an X-ray diffraction pattern comprises at least the following reflections:

| Diffraction angle 2Theta/° | |
|---|---|
| [Cu K (alpha 1)] | Intensity (%) |
| 8.5 to 10.5 | 90 to 100 |
| 15.1 to 17.1 | 75 to 95 |
| 15.9 to 17.9 | 80 to 100 |
| 16.2 to 18.2 | 80 to 100 |
| 19.7 to 21.7 | 80 to 100 |
| 20.4 to 22.4 | 50 to 70 |
| 23.2 to 25.2 | 80 to 100 |
| 25.3 to 27.3 | 30 to 50 |
| 30.2 to 32.2 | 40 to 60 |

wherein 100% relates to the intensity of the maximum peak in the X-ray powder diffraction pattern. More preferably, the zeolitic material having a framework type AEI has an X-ray diffraction pattern comprises at least the following reflections:

| Diffraction angle 2Theta/° | |
|---|---|
| [Cu K (alpha 1)] | Intensity (%) |
| 9.0 to 10.0 | 90 to 100 |
| 15.6 to 16.6 | 75 to 95 |
| 16.4 to 17.4 | 80 to 100 |
| 16.7 to 17.7 | 80 to 100 |
| 20.2 to 21.2 | 80 to 100 |
| 20.9 to 21.9 | 50 to 70 |
| 23.7 to 24.7 | 80 to 100 |
| 25.8 to 26.8 | 30 to 50 |
| 30.7 to 31.7 | 40 to 60 |

wherein 100% relates to the intensity of the maximum peak in the X-ray powder diffraction pattern.

Preferably. the porous oxidic material additionally comprises one or more transition metals.

more preferably one or more of Cu and Fe, more preferably Cu. More preferably, the porous oxidic material contains the one or more transition metals in a total amount of from 1.5 to 5.0 weight-%, more preferably of from 2.5 to 4.5 weight-%, more preferably of from 3.0 to 4.0 weight-%, based on the total weight of the porous oxidic material, calculated as elemental transition metal.

The present invention further relates to a use of a porous oxidic material according to the present invention as a catalytically active material, as a catalyst, or as a catalyst component. Preferably, the use is for the selective catalytic reduction of nitrogen oxides in an exhaust gas stream of a diesel engine. Alternatively, the use is preferably for converting methanol to one or more olefins.

The present invention further relates to a method for selectively catalytically reducing nitrogen oxides in an exhaust gas stream of a diesel engine, said method comprising preparing a porous oxidic material according to a process according to the present invention, preparing a catalyst comprising said porous oxidic material, and bringing said exhaust gas stream in contact with said catalyst.

The present invention further relates to a method for selectively catalytically reducing nitrogen oxides in an exhaust gas stream of a diesel engine, said method comprising preparing a catalyst comprising a porous oxidic material according to the present invention, and bringing said exhaust gas stream in contact with said catalyst.

The present invention further relates to a catalyst, preferably for selectively catalytically reducing nitrogen oxides in an exhaust gas stream of a diesel engine, said catalyst comprising a porous oxidic material according to the present invention.

The present invention further relates to a catalyst, preferably for catalytically converting methanol to one or more olefins, said catalyst comprising a porous oxidic material according to the present invention.

The present invention further relates to a method for catalytically converting methanol to one or more olefins, the method comprising
(i) providing a catalyst, the catalyst comprising a porous oxidic material according to the pre sent invention, or a porous oxidic material prepared according to a process according to the present invention;
(ii) providing a gas stream comprising methanol;
(iii) contacting the gas stream provided in (ii) with the catalyst provided in (i) into a reactor, obtaining a reaction mixture comprising one or more olefins.

It is preferred that (i) further comprises pretreating the catalyst in a reactor in a gas stream comprising nitrogen.

It is preferred that the catalyst provided in (i) is prepared by tableting the porous oxidic material according to the present invention, or the porous oxidic material prepared according to a process according to the present invention.

It is preferred that pretreating is performed in the gas stream comprising nitrogen at a temperature in the range of from 300 to 700° C., more preferably in the range of from 400 to 600° C.

It is preferred that contacting according to (iii) is effected at a temperature in the range of from 200 to 750° C. more preferably in the range of from 250 to 600° C., more preferably in the range of from 300 to 400° C.

It is preferred that contacting according to (iii) is effected at a pressure of the gas stream in the range of from 0.75 to 5 bar, more preferably in the range of from 0.9 to 1.5 bar.

It is preferred that contacting according to (iii) is carried out at a weight hourly space velocity in the range of from 0.2 to 100 h⁻¹, more preferably in the range of from 0.3 to 20 h⁻¹, more preferably in the range of from 0.4 to 10 h⁻¹, more preferably in the range of from 0.5 to 2 h⁻¹.

It is preferred that the reactor is a fixed-bed reactor.

The present invention is illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3 and 4”.
1. A process for preparing a porous oxidic material which comprises micropores and mesopores and which comprises a zeolitic material having a framework type AEI and having a framework structure comprising a tetravalent element Y, a trivalent element X and oxygen, wherein said micropores have a pore diameter determined according to nitrogen adsorption-desorption at 77 K of less than 2 nm and wherein said mesopores have a pore diameter determined according to nitrogen adsorption-desorption at 77 K in the range of from 2 to 50 nm, said process comprising:
   (i) preparing a synthesis mixture comprising a zeolitic material having a framework type FAU and having a framework structure comprising the tetravalent element Y, the trivalent element X and oxygen, water, a source of base, a first organic structure directing agent being an AEI framework type structure directing agent, a second organic structure directing agent comprising a dimethyl-octadecyl[3-(trimethoxysilyl)propyl]ammonium cation containing compound, and seed crystals;
   (ii) subjecting the mixture obtained in (i) to hydrothermal crystallization conditions comprising a crystallization temperature in the range of from 90 to 200° C., obtaining a mother liquor comprising the porous oxidic material comprising said zeolitic material having a framework type AEI;
   wherein Y is one or more of Si, Sn, Ti, Zr and Ge;
   wherein X is one or more of Al, B, In and Ga.
2. The process of embodiment 1, wherein the AEI framework type structure directing agent comprises a quaternary phosphonium cation containing compound, wherein preferably the quaternary phosphonium cation containing compound is a R1R2R3R4P+-containing compound, wherein R₁, R₂, R₃, and R4 independently from one another stand for optionally substituted and/or optionally branched (C1-C6)alkyl, more preferably (C1-C5)alkyl, more preferably (C1-C4)alkyl, more preferably (C₂-C3)alkyl, and preferably for optionally substituted methyl or ethyl, more preferably R1, R2, R3, and R4 stand for optionally substituted ethyl, more preferably unsubstituted ethyl,
   a N,N-diethyl-2,6-dimethylpiperidinium cation containing compound, a N,N-diethyl-3,5-dimethylpiperidinium cation containing compound, a N,N-dimethyl-2,6-dimethylpiperidinium cation containing compound, a N,N-dimethyl-3,5-dimethylpiperidinium cation containing compound, a N,N,N-trimethyl-1-adamantamonium cation containing compound, cis-2,6-dimethylpiperidinium cation containing compound, cis-trans-3,5-dimethylpiperidinium cation containing compound, a 2,2,7,7-tetramethyl-2-azabicyclo[4.1.1]octan-2-ium cation containing compound, a 1,3,3,6,6-pentamethyl-6-azabicyclo[3.2.1]octan-6-ium cation containing compound, or mixture thereof;
   wherein the AEI framework type structure directing agent preferably comprises a quaternary phosphonium cation containing compound, a N,N-diethyl-2,6-dimethylpiperidinium cation containing compound, a N,N-diethyl-3,5-dimethylpiperidinium cation containing compound, or mixture thereof;
   wherein the AEI framework type structure directing agent more preferably comprises a N,N-diethyl-2,6-dimethylpiperidinium cation containing compound, wherein the N,N-diethyl-2,6-dimethylpiperidinium cation containing compound is one or more of a N,N-diethyl-trans-2,6-dimethylpiperidinium cation containing compound and a N,N-diethyl-cis-2,6-dimethylpiperidinium cation containing compound, more preferably a N,N-diethyl-cis-2,6-dimethylpiperidinium cation containing compound.
3. The process of embodiment 2, wherein the N,N-diethyl-2,6-dimethylpiperidinium cation containing compound is a salt, preferably one or more of a hydroxide and a halide, preferably one or more of an iodide, a chloride, a fluoride and a bromide, wherein more preferably the N,N-diethyl-2,6-dimethylpiperidinium cation containing compound comprises, more preferably is, a hydroxide.
4. The process of any one of embodiments 1 to 3, wherein the dimethyloctadecyl[3-(tri-methoxysilyl)propyl]ammonium cation containing compound is a salt, preferably one or more of a hydroxide arid a halide, more preferably a halide, more preferably one or more of an iodide, a chloride, a fluoride and a bromide, wherein more preferably the dimethyloctadecyl[3-(trimethoxysilyl)propyl]ammonium cation containing compound comprises, more preferably is, a chloride.
5. The process of any one of embodiments 1 to 4, wherein from 95 to 100 weight-%, preferably from 98 to 100 weight-%, more preferably from 99 to 100 weight-%, more preferably from 99.5 to 100 weight-%, more preferably from 99.9 to 100 weight-%, of the porous oxidic material consist of the zeolitic material having a framework type AEI.
6. The process of any one of embodiments 1 to 4, wherein the porous oxidic material consists of micropores, mesopores and the zeolitic material having a framework type AEI.
7. The process of any one of embodiments 1 to 6, wherein Y is Si.
8. The process of any one of embodiments 1 to 7, wherein X is one or more of Al and B, preferably Al.
9. The process of any one of embodiments 1 to 8, wherein Y is Si and X is Al.
10. The process of any one of embodiments 1 to 9, wherein the zeolitic material provided in (i) and having a framework type FAU is a zeolitic material selected from the group consisting of a faujasite zeolite, a zeolite Y, a zeolite X, an LSZ-210 zeolite, a zeolite US Y, and a mixture of two or more thereof, preferably selected from the group consisting of zeolite Y, US Y and a mixture thereof, wherein more preferably the zeolitic material provided in (i) and having a framework type FAU is a zeolite US Y, or wherein more preferably the zeolitic material provided in (i) and having a framework type FAU is a zeolite Y.
11. The process of any one of embodiments 1 to 10, wherein in the framework structure of the zeolitic material having a framework type FAU provided in (i), the molar ratio of Y:X, calculated as YO₂:X₂O₃, is in the range of from 5:1 to 100:1, preferably in the range of from 10:1 to 60:1, more preferably in the range of from 18:1 to 45:1, more preferably in the range of from 20:1 to 37:1, more preferably in the range of from 20:1 to 30:1.
12. The process of any one of embodiments 1 to 11, wherein in the synthesis mixture in (i), the molar ratio of the first organic structure directing agent, FOSDA, relative to Y, calculated as FOSDA:YO₂, is in the range of from 0.05:1 to 0.30:1, preferably in the range of from 0.10:1 to 0.20:1.
13. The process of any one of embodiments 1 to 12, wherein in the synthesis mixture in (i), the molar ratio of the second organic structure directing agent, SOSDA, relative to Y, calculated as SOSDA:YO₂, is in the range of from 0.001:1 to 0.070:1, preferably in the range of from 0.002:1 to 0.060:1.
14. The process of embodiment 13, wherein in the synthesis mixture in (i), the molar ratio of the second organic structure directing agent, SOSDA, relative to Y, calculated as SOSDA:YO₂, is in the range of from 0.002:1 to 0.012:1, preferably in the range of from 0.004:1 to 0.011:1, more preferably in the range of from 0.006:1 to 0.010:1, more preferably in the range of from 0.007:1 to 0.009:1.
15. The process of embodiment 13, wherein in the synthesis mixture in (i), the molar ratio of the second organic structure directing agent, SOSDA, relative to Y, calculated as SOSDA:YO₂, is in the range of from 0.006:1 to 0.022:1, preferably in the range of from 0.010:1 to 0.020:1, more preferably in the range of from 0.013:1 to 0.018:1, more preferably in the range of from 0.015:1 to 0.017:1.
16. The process of embodiment 13, wherein in the synthesis mixture in (i), the molar ratio of the second organic structure directing agent, SOSDA, relative to Y, calculated as SOSDA:YO₂, is in the range of from 0.018:1 to 0.040:1, preferably in the range of from 0.021:1 to 0.028:1, more preferably in the range of from 0.023:1 to 0.026:1.
17. The process of embodiment 13, wherein in the synthesis mixture in (i), the molar ratio of the second organic structure directing agent, SOSDA, relative to Y, calculated as SOSDA:YO₂, is in the range of from 0.007:1 to 0.026:1 or is in the range of from 0.007:1 to 0.017:1.
18. The process of any one of embodiments 1 to 17, wherein in the synthesis mixture in (i), the molar ratio of the source of a base relative to Y, calculated as a source of a base:YO₂, is in the range of from 0.10:1 to 0.70:1, preferably in the range of from 0.20:1 to 0.60:1, more preferably in the range of from 0.30:1 to 0.55:1,
   wherein in the synthesis mixture in (i), the molar ratio of the source of a base relative to Y, calculated as a source of a base:YO₂, is more preferably in the range of from 0.39:1 to 0.50:1, more preferably in the range of from 0.40:1 to 0.48:1, more preferably in the range of from 0.44:1 to 0.47:1, or
   wherein in the synthesis mixture in (i), the molar ratio of the source of a base relative to Y, calculated as a source of a base:YO₂, is more preferably in the range of from 0.30:1 to 0.38:1, more preferably in the range of from 0.32:1 to 0.36:1.
19. The process of any one of embodiments 1 to 18, wherein in the synthesis mixture in (i), the molar ratio of H70 relative to Y, calculated as H₂O:YO₂, is in the range of from 2:1 to 80:1, preferably in the range of from 10:1 to 60:1, more preferably in the range of from 25:1 to 50:1, more preferably in the range of from 28:1 to 47:1, more preferably in the range of from 30:1 to 45:1.
20. The process of any one of embodiments 1 to 19, wherein the source of a base provided in (i) comprises, preferably is, a hydroxide.

21. The process of any one of embodiments 1 to 20, wherein the source of a base provided in (i) comprises, preferably is, one or more of an alkali metal hydroxide and an alkaline earth metal hydroxide, preferably an alkali metal hydroxide, more preferably sodium hydroxide.
22. The process of any one of embodiments 1 to 21, wherein the seed crystals provided in (i) comprise, preferably consist of, a zeolitic material having a framework type selected from the group consisting of AEI. CHA and RTH, preferably a zeolitic material having a framework type selected from the group consisting of AEI and CHA, wherein more preferably the seed crystals provided in (i) comprise, preferably consist of, a zeolitic material having a framework type AEI.
23. The process of any one of embodiments 1 to 22, wherein in the synthesis mixture (i), the weight ratio of the seed crystals to the zeolitic material having a framework type FAU is in the range of from 0.001:1 to 0.1:1, preferably in the range of from 0.005:1 to 0.05:1, more preferably in the range of from 0.01:1 to 0.04:1.
24. The process of any one of embodiments 1 to 23, wherein from 95 to 100 weight-%, preferably from 98 to 100 weight-%, more preferably from 99 to 100 weight-%, more preferably from 99.5 to 100 weight-% of the synthesis mixture consist of the zeolitic material having a framework type FAU and having a framework structure comprising the tetravalent element Y, the trivalent element X and oxygen, water, the source of base, the first organic structure directing agent comprising an AEI framework type structure directing agent, the second organic structure directing agent comprising a dimethyloctadecyl[3-(trimethoxysilyl)propyl]ammonium cation containing compound and the seed crystals.
25. The process of any one of embodiments 1 to 24, wherein preparing the synthesis mixture in (i) comprises
   (i.1) preparing a first mixture comprising the zeolitic material having a framework type FAU and having a framework structure comprising the tetravalent element Y, the trivalent element X and oxygen, water, and the first organic structure directing agent comprising an AEI framework type structure directing agent;
   (1.2) adding the source of base to the first mixture obtained in (1.1), obtaining a second mixture;
   (i.3) adding the second organic structure directing agent comprising a dimethyloctade-cyl[3-(trimethoxysilyl)propyl]ammonium cation containing compound to the second mixture obtained in (i.2), obtaining a third mixture;
   (i.4) adding the seed crystals to the third mixture obtained in (i.3), obtaining the synthesis mixture.
26. The process of embodiment 25, wherein preparing the first mixture in (i.1) comprises adding the first organic structure directing agent dropvvise to the zeolitic material.
27. The process of embodiment 25 or 26, wherein preparing the first mixture in (i.1) comprises agitating, preferably mechanically agitating, more preferably stirring, the mixture.
28. The process of embodiment 27, wherein agitating is performed at a temperature of the mixture in the range of from 12 to 35° C., preferably in the range of from 15 to 30° C.
29. The process of embodiment 27 or 28, wherein agitating is performed for a duration in the range of from 0.10 to 3 hours, preferably in the range of from 0.25 to 2 hours, more preferably in the range of from 0.4 to 1.75 hours, more preferably in the range of from 0.5 to 1.5 hours.
30. The process of any one of embodiments 25 to 29, wherein preparing the second mixture according to (i.2) comprises agitating, preferably mechanically agitating, more preferably stirring, the mixture.
31. The process of embodiment 30, wherein agitating is performed at a temperature of the mixture in the range of from 12 to 35° C., preferably in the range of from 15 to 30° C.
32. The process of embodiment 30 or 31, wherein agitating is performed for a duration in the range of from 0.10 to 3 hours, preferably in the range of from 0.25 to 2 hours, more preferably in the range of from 0.4 to 1.75 hours, more preferably in the range of from 0.5 to 1.5 hours.
33. The process of any one of embodiments 25 to 32, wherein preparing the third mixture according to (i.3) comprises agitating, preferably mechanically agitating, more preferably stirring, the mixture.
34. The process of embodiment 33, wherein agitating is performed at a temperature of the mixture in the range of from 12 to 35° C., preferably in the range of from 15 to 30° C.
35. The process of embodiment 33 or 34, wherein agitating is performed for a duration in the range of from 0.25 to 10 hours;
   wherein agitating is preferably performed for a duration in the range of from 0.25 to 4 hours, more preferably in the range of from 0.5 to 3 hours, more preferably in the range of from 1 to 2 hours; or
   wherein agitating is preferably performed for a duration in the range of from 1 to 8 hours, more preferably in the range of from 2 to 6 hours, more preferably in the range of from 3 to 5 hours.
36. The process of any one of embodiments 25 to 35, wherein preparing the synthesis mixture according to (i.4) comprises agitating, preferably mechanically agitating, more preferably stirring, the mixture.
37. The process of embodiment 36, wherein agitating is performed at a temperature of the mixture in the range of from 12 to 35° C., preferably in the range of from 15 to 30° C.
38. The process of embodiment 36 or 37, wherein agitating is performed for a duration in the range of from 5 to 50 minutes, preferably in the range of from 10 to 30 minutes, more preferably in the range of from 15 to 25 minutes, or preferably in the range of from 5 to 13 minutes.
39. The process of any one of embodiments 1 to 38, wherein the hydrothermal crystallization conditions according to (ii) comprise a crystallization duration in the range of from 0.75 to 20 days, preferably in the range of from 0.9 to 15 days, more preferably in the range of from 1 to 12 days, more preferably in the range of from 2 to 10 days, more preferably in the range of from 2 to 8 days, more preferably in the range of from 2 to 3.5 days or more preferably in the range of from 4 to 8 days.
40. The process of any one of embodiments 1 to 39, wherein the hydrothermal crystallization conditions according to (ii) comprise a crystallization temperature in the range of from 100 to 180° C., preferably in the range of from 120 to 160° C., more preferably in the range of from 130 to 150° C.
41. The process of any one of embodiments 1 to 40, wherein during the hydrothermal crystallization according to (ii), the mixture obtained in (i) and subjected to (ii) is agitated, preferably mechanically agitated, more preferably stirred.
42. The process of any one of embodiments 1 to 41, wherein according to (ii), subjecting the synthesis mixture obtained in (i) to hydrothermal crystallization conditions is carried out under autogenous pressure, preferably in an autoclave.
43. The process of any one of embodiments 1 to 42 further comprising
(iii) cooling the mother liquor comprising the porous oxidic material comprising the zeolitic material having a framework type AEI obtained from (ii), preferably to a temperature in the range of from 10 to 50° C.
44. The process of any one of embodiments 1 to 43 further comprising
(iv) separating the porous oxidic material from the mother liquor obtained from (ii) or (iii), preferably from
45. The process of embodiment 44, wherein (iv) comprises
   (iv.1) subjecting the mother liquor obtained from (ii) or (iii), preferably from (iii), to a solid-liquid separation method, preferably comprising a filtration method;
   (iv.2) preferably washing the porous oxidic material obtained from (iv.1);
   (iv.3) drying the porous oxidic material obtained from (iv.1) or (iv.2), preferably from (iv.2).
46. The process of embodiment 45, wherein according to (iv.2), the porous oxidic material is washed with water, preferably with deionized water.
47. The process of embodiment 45 or 46, wherein according to (iv.3), the porous oxidic material is dried in a gas atmosphere having a temperature in the range of from 60 to 200° C., preferably in the range of from 80 to 140° C., more preferably in the range of from 90 to 110° C.
48. The process of any of embodiments 45 to 47, wherein according to (iv.3), the porous oxidic material is dried in a gas atmosphere for a duration in the range of from 0.5 to 5 hours, preferably in the range of from 1 to 4 hours, more preferably in the range of from 1 to 3 hours.
49. The process of embodiment 47 or 48, wherein the gas atmosphere comprises, more preferably is, one or more of air, lean air, and oxygen, more preferably air.
50. The process of any one of embodiments 44 to 49 further comprising
   (v) calcining the porous oxidic material obtained from (iv), preferably from (iv.3), in a gas atmosphere.
51. The process of embodiment 50, wherein according to (v), the porous oxidic material is calcined in a gas atmosphere having a temperature in the range of from 300 to 550° C.
52. The process of embodiment 50 or 51, wherein according to (v), the porous oxidic material obtained from calcination has a total organic carbon content of at most 0.1 weight-%.
53. The process of any one of embodiments 50 to 52, wherein the gas atmosphere is air.
54. The process of any one of embodiments 1 to 53, wherein said micropores have a micropore volume and said mesopores have a mesopore volume and wherein the ratio of mesopore volume to micropore volume of the porous oxidic material is of at least 0.5:1 and the ratio of mesopore volume to the total pore volume of the porous oxidic material s of at least 0.3:1.
55. The process of any one of embodiments 1 to 54, wherein said micropores have a micropore volume and said mesopores have a mesopore volume and wherein the ratio of mesopore volume to micropore volume of the porous oxidic material is in the range of from 0.5:1 to 3:1;
   wherein the ratio of mesopore volume to micropore volume of the porous oxidic material is preferably in the range of from 0.75:1 to 2.5:1, more preferably in the range of from 1:1 to 2.1:1, more preferably in the range of from 1.35:1 to 2:1; or
   wherein the ratio of mesopore volume to micropore volume of the porous oxidic material is preferably in the range of from 0.55:1 to 2:1, more preferably in the range of from 0.6:1 to 1.25:1.
56. The process of any one of embodiments 1 to 55, wherein said micropores have a micropore volume and said mesopores have a mesopore volume and wherein the ratio of mesopore volume to the total pore volume of the porous oxidic material is in the range of from 0.3:1 to 1:1, preferably in the range of from 0.35:1 to 0.95:1;
   wherein the ratio of mesopore volume to the total pore volume of the porous oxidic material is more preferably in the range of from 0.4:1 to 0.9:1, more preferably in the range of from 0.50:1 to 0.75:1, more preferably in the range of 0.55:1 to 0.7:1; or
   wherein the ratio of mesopore volume to the total pore volume of the porous oxidic material is more preferably in the range of from 0.35:1 to 0.6:1, more preferably in the range of from 0.38:1 to 0.55:1.
57. The process of any one of embodiments 1 to 56, wherein said mesopores of the porous oxidic material have a mesopore volume, determined as described in Reference Example 1 b), in the range of from 0.15 to 0.80 cm³/g.
58. The process of embodiment 57, wherein the mesopore volume, determined as described in Reference Example 1 b), is in the range of from 0.20 to 0.65 cm³/g, preferably in the range of from 0.25 to 0.55 cm³/g, more preferably in the range of from 0.30 to 0.50 cm³/g.
59. The process of embodiment 57 or 58, wherein the mesopore volume, determined as described in Reference Example 1 b), is in the range of from 0.30 to 0.40 cm³/g, more preferably in the range of from 0.32 to 0.38 cm³/g; or wherein the mesopore volume, determined as described in Reference Example 1 b), is in the range of from 0.40 to 0.50 cm³/g, more preferably in the range of from 0.42 to 0.48 cm³/g.
60. The process of embodiment 57, wherein the mesopore volume, determined as described in Reference Example 1 b), is in the range of from 0.15 to 0.50 cm³/g, preferably in the range of from 0.15 to 0.40 cm³/g, more preferably in the range of from 0.16 to 0.30 cm³/g.

61. The process of any one of embodiments 1 to 60, wherein said micropores of the porous oxidic material have a micropore volume, determined as described in Reference Example 1 b), in the range of from 0.05 to 0.50 cm³/g, preferably in the range of from 0.10 to 0.40 cm³/g, more preferably in the range of from 0.20 to 0.30 cm³/g.
62. The process of any one of embodiments 44 to 61 further comprising
   (vi) subjecting the porous oxidic material obtained from (iv) or (v), preferably from (iv.3) or (v), to ion-exchange conditions.
63. The process of embodiment 62, wherein (vi) comprises
   (vi.1) subjecting the porous oxidic material obtained from (iv) or (v), preferably from (iv.3) or (v), to ion-exchange conditions comprising bringing a solution comprising ammonium ions in contact with the porous oxidic material obtained from (iv) or (v), obtaining a porous oxidic material in its ammonium form;
   wherein the solution comprising ammonium ions according to (vi.1) is preferably an aqueous solution comprising a dissolved ammonium salt, preferably a dissolved inorganic ammonium salt, more preferably a dissolved ammonium nitrate.
64. The process of embodiment 63, wherein the solution comprising ammonium ions according to (vi.1) has an ammonium concentration in the range of from 0.10 to 3 moi/l, preferably in the range of from 0.20 to 2 mol/l, more preferably in the range of from 0.5 to 1.5 mol/l.
65. The process of embodiment 63 or 64, wherein according to (vi.1), the solution comprising ammonium ions is brought in contact with the zeolitic material obtained from (iv) or (v) at a temperature of the solution in the range of from 60 to 100° C., preferably in the range of from 70 to 90° C.
66. The process of any one of embodiments 63 to 65, wherein according to (vi.1), the solution comprising ammonium ions is brought in contact with the zeolitic material obtained from (iv) or (v) for a period of time in the range of from 1 to 6 hours, preferably in the range of from 1.5 to 4 hours.
67. The process of any one of embodiments 63 to 66, wherein bringing the solution in contact with the porous oxidic material according to (vi.1) comprises one or more of impregnating the porous oxidic material with the solution and spraying the solution onto the porous oxidic material, preferably impregnating the porous oxidic material with the solution.
68. The process of any one of embodiments 63 to 67, wherein (vi) further comprises
   (vi.2) calcining the porous oxidic material obtained in (vi.1) in a gas atmosphere, preferably in a gas atmosphere having a temperature in the range of from 450 to 650° C., more preferably in the range of from 500 to 600° C., obtaining the H-form of the porous oxidic material.
69. The process of embodiment 68, wherein calcining according to (iv.2) is performed in gas atmosphere for a duration in the range of from 2 to 6 hours, preferably in the range of from 3 to 5 hours,
70. The process of embodiment 68 or 69, wherein vi.1) and (vi.2) are carried out at least once, preferably twice.
71. The process of any one of embodiments 68 to 70, wherein the gas atmosphere comprises, preferably is, one or more of air, lean air, and oxygen, more preferably air.
72. The process of any one of embodiments 68 to 71, wherein (vi) further comprises (vi.3) subjecting the porous oxidic material obtained from (vi.2) to ion-exchange conditions comprising bringing a solution comprising ions of one or more transition metals, preferably of one or more of Cu and Fe, more preferably Cu, in contact with the porous oxidic material obtained from (vi.2).
73. The process of embodiment 72, wherein the solution comprising ions of one or more transition metals according to (vi.3) is an aqueous solution comprising a dissolved salt of one or more transition metals, preferably a dissolved organic copper salt, more preferably a dissolved copper acetate.
74. The process of embodiment 72 or 73, wherein the solution comprising ions of one or more transition metals according to (vi.3) has a transition metal concentration, preferably a copper concentration, in the range of from 0.10 to 3 mol/l, preferably in the range of from 0.20 to 2 mol/l, more preferably in the range of from 0.5 to 1.5 mol/l.
75. The process of any one of embodiments 72 to 74, wherein according to (vi.3), the solution comprising ions of one or more transition metals is brought in contact with the porous oxidic material obtained from (vi.2) at a temperature of the solution in the range of from 60 to 100° C., preferably in the range of from 70 to 90° C.
76. The process of any one of embodiments 72 to 75, wherein according to (vi.3), the solution comprising ions of one or more transition metals is brought in contact with the zeolitic material obtained from (vi.2) for a period of time in the range of from 0.5 to 3 hours, preferably in the range of from 0.5 to 2 hours.
77. The process of any one of embodiments 72 to 76, wherein (vi) further comprises (vi.4) calcining the porous oxidic material obtained in (vi.3) in a gas atmosphere, preferably in a gas atmosphere having a temperature in the range of from 450 to 650° C., more preferably in the range of from 500 to 600° C.
78. The process of embodiment 77, wherein calcining according to (vi.4) is performed in gas atmosphere for a duration in the range of from 1 to 6 hours, preferably in the range of from 3 to 5 hours.
79. The process of embodiment 77 or 78, wherein the gas atmosphere comprises, preferably is, one or more of air, lean air, arid oxygen, more preferably air.
80. The process of any one of embodiments 68 to 79 further comprising (vii) ageing the porous oxidic material obtained in (vi.2), preferably in (vi.4), in gas atmosphere.
81. The process of embodiment 80, wherein ageing in (vii) is performed in gas atmosphere, preferably in air, having a temperature in the range of from 400 to 1000° C., preferably in the range of from 600 to 800° C.
82. The process of embodiment 80 or 81, wherein ageing in (vii) is performed for a duration in the range of from 5 to 100 hours, preferably in the range of from 10 to 60 hours.
83. The process of any one of embodiments 80 or 82, wherein the gas atmosphere comprises, preferably is, one or more of air, lean air, and oxygen, more preferably air.
84. A porous oxidic material which comprises micropores and mesopores and which comprises a zeolitic material having a framework type AEI and having a framework structure comprising a tetravalent element Y, a trivalent element X, arid oxygen, wherein said micropores have a pore diameter determined according to nitrogen adsorption-desorption at 77 K of less than 2 rim and wherein said mesopores have a pore diameter determined according to nitrogen adsorption-desorption at 77 K in the range of from 2 to 50 nm, wherein Y is one or more of Si, Sn, Ti, Zr, and Ge and wherein X is one or more of Al, B, In, and Ga, wherein said micropores have a micropore volume and said mesopores have a mesopore volume, the ratio of mesopore volume to micropore volume being of at least 0.5:1 and the ratio of mesopore volume to the total pore volume of the porous oxidic material being of at least 0.3:1, wherein the porous oxidic material is preferably obtainable or obtained by a process according to any one of embodiments 1 to 83.
85. The porous oxidic material of embodiment 84, wherein Y is Si and X is one or more of Al and B, more preferably wherein X is Si and X is Al.
86. The porous oxidic material of embodiment 84 or 85, wherein the zeolitic material having a framework type AEI is a zeolite SSZ-39.
87. The porous oxidic material of any one of embodiments 84 to 86, wherein from 92 to 100 weight-%, preferably from 95 to 100 weight-%, more preferably from 98 to 100 weight-%, more preferably from 99 to 100 weight-%, more preferably from 99.5 to 100 weight-%, more preferably from 99.9 to 100 weight-%, of the porous oxidic material consist of the zeolitic material having a framework type AEI and the mesopores.
88. The porous oxidic material of any one of embodiment 84 to 87, wherein in the framework structure of the zeolitic material having framework type AEI, the molar ratio of Y:X, calculated as a YO₂:X₂O₃, is in the range of from 2:1 to 40:1, preferably in the range of from 10:1 to 30:1, more preferably in the range of from 14:1 to 26:1, more preferably in the range of from 16:1 to 24:1.
89. The porous oxidic material of any one of embodiments 84 to 88, having a BET specific surface area, determined as described in Reference Example 1 b), in the range of from 500 to 900 m²/g;
   wherein the porous oxidic material preferably has a BET specific surface area, determined as described in Reference Example 1 b), in the range of from 520 to 600 m²/g, more preferably in the range of from 540 to 575 m²/g; or
   wherein the porous oxidic material preferably has a BET specific surface area, determined as described in Reference Example 1 b), in the range of from 600 to 900 m²/g, more preferably in the range of from 650 to 850 m²/g, more preferably in the range of from 750 to 830 m²/g, more preferably in the range of from 785 to 820 m²/g.
90. The porous oxidic material of any one of embodiments 84 to 89, wherein the mesopore volume, determined as described in Reference Example 1 b), is in the range of from 0.15 to 0.80 cm³/g.
91. The porous oxidic material of embodiment 90, wherein the mesopore volume, determined as described in Reference Example 1 b), is in the range of from 0.20 to 0.65 cm³/g, more preferably in the range of from 0.25 to 0.55 cm³/g, more preferably in the range of from 0.30 to 0.50 cm³/g.
92. The porous oxidic material of embodiment 90 or 91, wherein the mesopore volume, determined as described in Reference Example 1 b), is in the range of from 0.30 to 0.40 cm³/g, more preferably in the range of from 0.32 to 0.38 cm³/g; or
   wherein the mesopore volume, determined as described in Reference Example 1 b), is in the range of from 0.40 to 0.50 cm³/g, more preferably in the range of from 0.42 to 0.48 cm³/g.
93. The porous oxidic material of embodiment 90, wherein the mesopore volume, determined as described in Reference Example 1 b). is in the range of from 0.15 to 0.50 cm³/g, preferably in the range of from 0.15 to 0.40 cm³/g, more preferably in the range of from 0.16 to 0.30 cm³/g.
94. The porous oxidic material of any one of embodiments 84 to 93, wherein the micropore volume, determined as described in Reference Example 1 b), is in the range of from 0.05 to 0.50 cm³/g, preferably in the range of from 0.10 to 0.40 cm³/g, more preferably in the range of from 0.20 to 0.30 cm³/g.
95. The porous oxidic material of any one of embodiments 84 to 94, wherein the ratio of mesopore volume to micropore volume is in the range of from 0.5:1 to 3:1;
   wherein the ratio of mesopore volume to micropore volume of the porous oxidic material is preferably in the range of from 0.75:1 to 2.5:1, more preferably in the range of from 1:1 to 2.1:1, more preferably in the range of from 1.35:1 to 2:1; or
   wherein the ratio of mesopore volume to micropore volume of the porous oxidic material is preferably in the range of from 0.55:1 to 2:1, more preferably in the range of from 0.6:1 to 1.25:1.
96. The porous oxidic material of any one of embodiments 84 to 95, wherein the ratio of mesopore volume to the total pore volume is in the range of from 0.3:1 to 1:1, preferably in the range of from 0.35:1 to 0.95:1, more preferably in the range of from 0.4:1 to 0.9:1, more preferably in the range of from 0.50:1 to 0.75:1, more preferably in the range of from 0.55:1 to 0.7:1.
97. The porous oxidic material of any one of embodiments 84 to 96, having a crystallinity, determined as described in Reference Example 1 e), in the range of from 80 to 100%, preferably in the range of from 90 to 100%, more preferably in the range of from 99 to 100%.
98. The porous oxidic material of any one of embodiments 84 to 97, wherein the zeolitic material having a framework type AEI has an X-ray diffraction pattern comprising at least the following reflections:

| Diffraction angle 2Theta/° | |
|---|---|
| [Cu K (alpha 1)] | Intensity (%) |
| 8.5 to 10.5 | 90 to 100 |
| 15.1 to 17.1 | 75 to 95 |
| 15.9 to 17.9 | 80 to 100 |
| 16.2 to 18.2 | 80 to 100 |
| 19.7 to 21.7 | 80 to 100 |
| 20.4 to 22.4 | 50 to 70 |
| 23.2 to 25.2 | 80 to 100 |
| 25.3 to 27.3 | 30 to 50 |
| 30.2 to 32.2 | 40 to 60 |

wherein 100% relates to the intensity of the maximum peak in the X-ray powder diffraction pattern, preferably having a framework type AEI has an X-ray diffraction pattern comprising at least the following reflections:

| Diffraction angle 2Theta/° | |
|---|---|
| [Cu K (alpha 1)] | Intensity (%) |
| 9.0 to 10.0 | 90 to 100 |
| 15.6 to 16.6 | 75 to 95 |
| 16.4 to 17.4 | 80 to 100 |
| 16.7 to 17.7 | 80 to 100 |
| 20.2 to 21.2 | 80 to 100 |
| 20.9 to 21.9 | 50 to 70 |
| 23.7 to 24.7 | 80 to 100 |
| 25.8 to 26.8 | 30 to 50 |
| 30.7 to 31.7 | 40 to 60 |

wherein 100% relates to the intensity of the maximum peak in the X-ray powder diffraction pattern.
99. The porous oxidic material of any one of embodiments 84 to 98, additionally comprising one or more transition metals, preferably one or more of Cu and Fe, more preferably Cu; wherein the porous oxidic material preferably contains the one or more transition metals in a total amount of from 1.5 to 5.0 weight-%, preferably of from 2.5 to 4.5 weight-%, more preferably of from 3.0 to 4.0 weight-%, based on the total weight of the porous oxidic material, calculated as elemental transition metal.
100. Use of a porous oxidic material according to any one of embodiments 84 to 99 as a catalytically active material, as a catalyst, or as a catalyst component.
101. The use of embodiment 100 for the selective catalytic reduction of nitrogen oxides in an exhaust gas stream of a diesel engine.
102. The use of embodiment 100 for converting methanol to one or more olefins.
103. A method for selectively catalytically reducing nitrogen oxides in an exhaust gas stream of a diesel engine, said method comprising preparing a porous oxidic material according to a process according to any one of embodiments 1 to 83, preparing a catalyst comprising said porous oxidic material, and bringing said exhaust gas stream in contact with said catalyst.
104. A method for selectively catalytically reducing nitrogen oxides in an exhaust gas stream of a diesel engine, said method comprising preparing a catalyst comprising a porous oxidic material according to any one of embodiments 84 to 99, and bringing said exhaust gas stream in contact with said catalyst.
105. A catalyst, preferably for selectively catalytically reducing nitrogen oxides in an exhaust gas stream of a diesel engine, said catalyst comprising a porous oxidic material according to any one of embodiments 84 to 99.
106. A catalyst, preferably for catalytically converting methanol to one or more olefins, said catalyst comprising a porous oxidic material according to any one of embodiments 84 to 99.
107. A method for catalytically converting methanol to one or more olefins, the method comprising
   (i) providing a catalyst, the catalyst comprising a porous oxidic material according to any one of embodiments 84 to 99, or a porous oxidic material prepared according to a process of any one of embodiments 1 to 83;
   (ii) providing a gas stream comprising methanol;
   (iii) contacting the gas stream provided in (ii) with the catalyst provided in (i) into a reactor, obtaining a reaction mixture comprising one or more olefins.
108. The method of embodiment 107, wherein (i) further comprises pretreating the catalyst in a reactor in a gas stream comprising nitrogen.
109. The method of embodiment 108, wherein pretreating is performed in the gas stream comprising nitrogen at a temperature in the range of from 300 to 700° C., preferably in the range of from 400 to 600° C.
110. The method of any one of embodiments 107 to 109, wherein contacting according to (iii) is effected at a temperature in the range of from 200 to 750° C., preferably in the range of from 250 to 600° C., more preferably in the range of from 300 to 400° C.
111. The method of any one of embodiments 107 to 110, wherein contacting according to (iii) is effected at a pressure of the gas stream in the range of from 0.75 to 5 bar, preferably in the range of from 0.9 to 1.5 bar.
112. The method of any one of embodiments 107 to 111, wherein contacting according to (iii) is carried out at a weight hourly space velocity in the range of from 0.2 to 100 h⁻¹, preferably in the range of from 0.3 to 20 h⁻¹, more preferably in the range of from 0.4 to 10 h⁻i, more preferably in the range of from 0.5 to 2 h⁻¹.
113. The method of any one of embodiments 107 to 112, wherein the reactor is a fixed-bed reactor.

The present invention is further illustrated by the following examples, reference examples, and comparative examples.

### EXAMPLES

### Reference Example 1

### Characterizations

a) X-ray powder diffraction (XRD) patterns were measured with Rigaku Ultimate VI X-ray diffractometer (40 kV, 40 mA) using Cu_{Kalpha} radiation (lambda=1.5406 Angstrom).
b) The N7 sorption isotherms at the temperature of liquid nitrogen were measured using Micromeritics ASAP 2020M (or FINESORB-3020M) and Tristar system for determining the BET specific surface area. The micropore volume and the mesopore volume were measured by BJH (Barett, Joyner, Halenda) analysis according to DIN 66134.
c) The sample composition was determined by inductively coupled plasma (ICP) with a Perkin-Elmer 3300 DV emission spectrometer.
d) Scanning electron microscopy (SEM) experiments were performed on Hitachi SU-1510 electron microscopes or on Hitachi SU-8010 electron microscopes.
e) The crystallinity was calculated with the intensity of the peak with the highest intensity of a sample on the X-ray powder diffraction (XRD) pattern measured as in a) versus the intensity of the peak with the highest intensity of a fully crystallized sample.
f) Elemental analyses were tested by Elementar Vario MICRO cube.
g) The acidity of the catalysts was measured by the temperature-programmed-desorption of ammonia (NH₃-TPD). The catalyst was prepared at 450° C. in a He flow for 1 h, followed by the adsorption of NH₃ at 100° C. for 1 h. After saturation, the catalyst was purged by a He flow for 3 h to remove the physically adsorbed ammonia on the sample. Then, desorption of NH₃ was carried out from 100 to 600° C. with a heating rate of 10° C./min. The amount of NH₃ desorbed from the sample was detected using a thermal conductivity detector.
h) The thermogravimetry-differential thermal analysis (TG-DTA) experiments were carried out on a Perkin-Elmer TGA 7 unit in air at heating rate of 10° C./min in the temperature range from room temperature to 800° C.

### Reference Example 2

### Preparation of N,N-diethyl-cis-2,6-dimethylpiperidinium hydroxide as an Organic Structure Directing Agent (FOSDA)

36 g of cis-2,6-dimethylpiperidine (99%, Tokyo Chemical Industry Co., LTD.), 200 g of iodoethane (CH₃CH₂I, 99%, Aladdin Industrial Co.), 64 g of potassium bicarbonate (99.5%, Sinopharm Chemical Reagent Co., Ltd) were dissolved in 100 g of methanol anhydrous (99.5 /0, Sinopharm Chemical Reagent Co., Ltd) and heated under reflux at 50° C. for 4 days. The solvent and the excess of iodoethane were removed using rotary evaporation. The solid product was washed twice with 200 mL of trichloromethane (99%, Sinopharm Chemical Reagent Co., Ltd) each time. The resulting product was added in 20 mL of ethyl alcohol. 500 mL of diethyl ether anhydrous (99.7%, Sinopharm Chemical Reagent Co., Ltd) was further added. The mixture was stirred for 1 hour. The mixture was filtered and the solid product was collected.

Finally, 29.7 g of the solid product was mixed with 75 g of H₂O and 110 g of Amberlite IRN-78 ion-exchange resin, OH⁻form, and stirred for 1 day. The mixture was filtered and the solution obtained is the hydroxide form of N,N-diethyl-cis-2,6-dimethylpiperidinium.

### Example 1

### Preparation of a Mesoporous Zeolitic Material having a Framework Type AEI

a) Preparing a mesoporous zeolitic material having a framework type AEI

Materials:

| | | |
|---|---|---|
| Zeolite Y (USY) powder having a SiO₂:Al₂O₃ molar | 1 | g |
| ratio of 24:1 | | |
| N,N-diethyl-cis-2,6-dimethylpiperidinium hydroxide solution | 11.04 | g |
| (0.21M) as obtained in Reference Example 2 | | |
| NaOH powder | 0.29 | g |
| Dimethyloctadecyl[3-(trimethoxysilyl)propyl]ammonium | 0.20 | g |
| chloride (65% concentration) | | |
| Seed crystals AEI (not mesoporous) | 0.02 | g |

11.04 g of N,N-diethyl-cis-2,6-dimethylpiperidinium hydroxide solution (0.21 M) (the first structure directing agent, FOSDA) was added dropwise to 1 g of zeolite Y in a beaker and the mixture was stirred magnetically for 1 hour at room temperature. 0.29 g of NaOH was added and the mixture was stirred magnetically for 1 hour.

0.20 g of dimethyloctadecyl[3-(trimethoxysilyl)propyl]ammonium chloride (the second structure directing agent, SOSDA) was added to the mixture and the resulting mixture was stirred magnetically for 1.5 hours at room temperature. Finally, 0.02 g of AEI seed crystals was added and the mixture was stirred for 20 minutes at room temperature. The synthesis mixture composition was 0.23 Na₂O: 0.15 FOSDA: 0.016 SOSDA: 1.0 SiO₂: 0.042 Al₂O₃: 37.5 H₂O. The term SiO₂ refers to the silicon comprised in the zeolite Y calculated as silica. The obtained mixture was transferred in a Teflon-lined autoclave with a volume of 25 ml. The autoclave was sealed and the mixture crystallized at 140° C. for 7 days (168 hours) under rotation (at a constant speed of 50 rpm). After pressure release and cooling to room temperature, the obtained suspension was subjected to filtration. The filter cake was washed with deionized water and was then dried for 2 hours at a temperature of 100° C. 0.80 g of a zeolitic material was obtained. The SiO₂: Al₂O₃ molar ratio of the zeolitic material was 20.

The XRD patterns of the respectively obtained zeolitic material show the characteristic peaks of AEI framework structure, namely a peak at around 9.5 2Theta (highest intensity, 100%), a peak at around 16.1° 2Theta (intensity: 85.9%), a peak at around 16.9° 2Theta (intensity: 92.0%), a peak at around 17.2° 2Theta (intensity: 97.2%), a peak at around 20.7° 2Theta (intensity: 90.1%), a peak at around 21.4° 2Theta (intensity: 62.0%), a peak at 24.0° 2Theta (intensity: 90.8%), a peak at 26.3° 2Theta (intensity: 43.9%) and a peak at 31.2° 2Theta (intensity: 53.3%), as shown in FIG. 1.

The BET specific surface area of the respectively obtained zeolitic material, after calcination in air at 550° C. for 4 hours, was 796.3 m²/g determined as described in Reference Example 1 b).

The mesopore volume was of 0.36 cm³/g as determined as described in Reference Example 1 b) and the micropore volume was of 0.26 cm³/g as determined as described in Reference Example 1 b). The ratio of mesopore volume to micropore volume was 1.38:1 and the ratio of mesopore volume to the total pore volume was 0.581:1.

The SEM images of the respectively obtained fresh AEI zeolitic material (low magnification: scale bar 1 micrometer) are shown in FIG. 2a. The SEM images of the respectively obtained fresh AEI zeolitic material (high magnification: scale bar 500 nm) are shown in FIG. 2b. The crystallinity of the sample was of approximately 100%, determined as described in Reference Example 1 e), as shown in FIG. 3.

b) Preparing the H-form of a mesoporous zeolitic material having a framework type AEI

The dried zeolitic material obtained from a) is ion-exchanged with a 1M NH₄NO₃ solution at 80 ° C. for 2 hours and calcined in air at 550° C. for 4 hours. The procedure was repeated once.

c) Preparing the Cu-form of a mesoporous zeolitic material having a framework type AEI The H-form zeolitic material obtained from b) was ion-exchanged with 1 M Cu(CH₃COO)2 aqueous solution at 80° C. for 1 hour and calcined in air at 550° C. for 4 hours.

Copper content (Cu) of the Cu-exchanged AEI zeolitic material: 3.32 weight-%, calculated as elemental Cu, based on the total weight of the zeolitic material.

### Example 2

### Preparation of a Mesoporous Zeolitic Material having a Framework Type AEI (Varying the Ratio SOSDA: SiO₂)

a) Preparing a mesoporous zeolitic material having a framework type AEI Materials:

| | | |
|---|---|---|
| Zeolite Y (USY) powder having a SiO₂:Al₂O₃ molar | 1 | g |
| ratio of 24:1 | | |
| N,N-diethyl-cis-2,6-dimethylpiperidinium hydroxide solution | 11.04 | g |
| (0.21M) as obtained in Reference Example 2 | | |
| NaOH powder | 0.29 | g |
| Dimethyloctadecyl[3-(trimethoxysilyl)propyl]ammonium | 0.10 | g |
| chloride (65% concentration) | | |
| Seed crystals AEI (not mesoporous) | 0.02 | g |

11.04 g of N,N-diethyl-cis-2,6-dimethylpiperidinium hydroxide solution (0.21 M) (the first structure directing agent, FOSDA) was added dropwise to 1 g of zeolite Y in a beaker and the mixture was stirred magnetically for 1 hour at room temperature. 0.29 g of NaOH was added and the mixture was stirred magnetically for 1 hour.

0.10 g of dimethyloctadecyl[3-(trimethoxysilyl)propyl]ammonium chloride (the second structure directing agent, SOSDA) was added to the mixture and the resulting mixture was stirred magnetically for 1.5 hours at room temperature. Finally, 0.02 g of AEI seed crystals was added and the mixture was stirred for 20 minutes at room temperature. The synthesis mixture composition was 0.23 Na₂O: 0.15 FOSDA: 0.008 SOSDA: 1.0 SiO₂: 0.042 Al₂O₃: 37.5 H₂O. The term SiO₂ refers to the silicon comprised in the zeolite Y calculated as silica. The obtained mixture was transferred in a Teflon-lined autoclave with a volume of 25 ml. The autoclave was sealed and the mixture crystallized at 140° C. for 7 days (168 hours) under rotation (at a constant speed of 50 rpm). After pressure release and cooling to room temperature, the obtained suspension was subjected to filtration. The filter cake was washed with deionized water and was then dried for 2 hours at a temperature of 100° C. 0.80 g of a zeolitic material was obtained. The SiO₂: Al₂O₃ molar ratio of the zeolitic material was 20.

The XRD patterns of the respectively obtained zeolitic material show the characteristic peaks of AEI framework structure, namely a peak at around 9.5° 2Theta (highest intensity), a peak at around 16.1° 2Theta, a peak at around 16.9 2Theta, a peak at around 17.2 2Theta, a peak at around 20.7° 2Theta, a peak at around 21.4 2Theta, a peak at 24.0° 2Theta, a peak at 26.3 2Theta and a peak at 31.2° 2Theta, as shown in FIG. 1.

The BET specific surface area of the respectively obtained zeolitic material, after calcination in air at 550° C. for 4 hours, was 798.4 m²/g determined as described in Reference Example 1 b).

The mesopore volume was of 0.44 cm³/g as determined as described in Reference Example 1 b) and the micropore volume was of 0.23 cm³/g as determined as described in Reference Example 1 b). The ratio of the mesopore volume to the micropore volume was 1.91:1 and the ratio of the mesopore volume to the total pore volume was 0.657:1.

The SEM images of the respectively obtained fresh AEI zeolitic material (low magnification: scale bar 1 micrometer) are shown in FIG. 5a.The SEM images of the respectively obtained fresh AEI zeolitic material (high magnification: scale bar 500 nm) are shown in FIG. 5b. The crystallinity of the sample was of 100%, determined as described in Reference Example 1 e).

b) Preparing the H-form of a mesoporous zeolitic material having a framework type AEI The dried zeolitic material obtained from a) is ion-exchanged with a 1M NH₄NO₃ solution at 80° C. for 2 hours and calcined in air at 550° C. for 4 hours. The procedure was repeated once.

c) Preparing the Cu-form of a mesoporous zeolitic material having a framework type AEI The H-form zeolitic material obtained from b) was ion-exchanged with 1 M Cu(CH₃COO)₂ aqueous solution at 80° C. for 1 hour and calcined in air at 550° C. for 4 hours. Copper content (Cu) of the Cu-exchanged AEI zeolitic material: 3.46 weight-%, calculated as elemental Cu, based on the total weight of the zeolitic material.

### Comparative Example 1

### Attempt to Prepare a Mesoporous Zeolitic Material having a Framework Type AEI in the Absence of dimethyloctadecyl[3-(trimethoxysilyl)propyl]ammonium chloride

a) Trying to prepare a mesoporous zeolitic material having a framework type AEI Materials:

| | | |
|---|---|---|
| Zeolite Y (USY) powder having a SiO₂:Al₂O₃ molar | 1 | g |
| ratio of 24:1 | | |
| N,N-diethyl-cis-2,6-dimethylpiperidinium hydroxide solution | 11.04 | g |
| (0.21M) as obtained in Reference Example 2 | | |
| NaOH powder | 0.29 | g |
| Seed crystals AEI (not mesoporous) | 0.02 | g |

11.04 g of N,N-diethyl-cis-2,6-dimethylpiperidinium hydroxide solution (0.21 M) (FOSDA) was added dropwise to 1 g of zeolite Y in a beaker and the mixture was stirred magnetically for 1 hour at room temperature. 0.29 g of NaOH was added and the mixture was stirred magnetically for 1 hour.

Finally, 0.02 g of AEI seed crystals was added and the mixture was stirred for 20 minutes at room temperature. The synthesis mixture composition was 0.23 Na₂O: 0.15 FOSDA: 1.0 SiO₂: 0.042 Al₂O₃: 37.5 H₂O. The term SiO₂ refers to the silicon comprised in the zeolite Y calculated as silica. The obtained mixture was transferred in a Teflon-lined autoclave with a volume of 25 ml. The autoclave was sealed and the mixture crystallized at 140° C. for 3 days (72 hours) under homogeneous rotation (50 rpm). After pressure release and cooling to room temperature, the obtained suspension was subjected to filtration. The filter cake was washed with deionized water and was then dried for 2 hours at a temperature of 100° C. 0.80 g of a zeolitic material was obtained. The SiO₂: Al₂O₃ molar ratio of the zeolitic material was 20.

The XRD patterns of the respectively obtained zeolitic material show the characteristic peaks of AEI framework structure, namely a peak at around 9.5° 2Theta (highest intensity), a peak at around 16.1° 2Theta, a peak at around 16.9° 2Theta, a peak at around 17.2° 2Theta, a peak at around 20.7° 2Theta, a peak at around 21.4° 2Theta, a peak at 24.0° 2Theta, a peak at 26.3° 2Theta and a peak at 31.2° 2Theta, as shown in FIG. 1.

After calcination in air at 550° C. for 4 hours, the BET specific surface area was 782.9 m²/g determined as described in Reference Example 1 b).

The mesopore volume was of 0.07 cm³/g determined as described in Reference Example 1 b) and the micropore volume was of 0.29 cm³/g determined as described in Reference Example 1 b). The ratio of the mesopore volume to the micropore volume was 0.241:1 and the ratio of the mesopore volume to the total pore volume was 0.194:1.

The SEM images of the respectively obtained fresh AEI zeolitic material (low magnification: scale bar 1 micrometer) are shown in FIG. 6a.The SEM images of the respectively obtained fresh AEI zeolitic material (high magnification: scale bar 500 nm) are shown in FIG. 6b. The crystallinity of the sample was of 100%, determined as described in Reference Example 1 e).

b) Preparing the H-form of a zeolitic material having a framework type AEI

The dried zeolitic material obtained from a) is ion-exchanged with a 1M NH₄NO₃ solution at 80° C. for 2 hours and calcined in air at 550° C. for 4 hours. The procedure was repeated once.

c) Preparing the Cu-form of a zeolitic material having a framework type AEI The H-form zeolitic material obtained from c) was ion-exchanged with 1 M Cu(CH₃COO)₂ aqueous solution at 80° C. for 1 hour and calcined in air at 550° C. for 4 hours. Copper content (Cu) of the Cu-exchanged AEI zeolitic material: 3.65 weight-%, calculated as elemental Cu, based on the total weight of the zeolitic material.

The zeolitic material obtained according to the synthesis of Comparative Example 1 a) and having a framework structure of the type AEI is not rnesoporous. In particular, this is illustrated by the measurements of the micropore volume and the mesopore volume. It results from said data that the mesopore volume is not significant compared to the total pore volume (mesopore volume+micropore volume) as it represents less than 20% of the total pores. Thus, Comparative Example 1 demonstrates that a dimethyloctadecyl[3-(trimethoxysilyl)propyl]ammonium containing compound is an essential compound for preparing a mesoporous zeolitic material having a framework type AEI.

### Example 4

### Use of Zeolitic Materials having a Framework Type AEI for Selectively Catalytitally Reducing Nitrogen Oxides

Catalysts comprising the zeolitic materials respectively obtained from Examples 1 and 2 and from Comparative Example 1 were prepared by tableting and squashing to 40-60 mesh and subjected to a selective catalytic reduction test. The amount of catalysts used in the fixed bed is 0.5 g each. For this purpose, the catalytic activities of the respectively obtained fresh catalysts were measured with a fixed-bed quartz reactor in a gaseous mixture containing 500 ppm of NO, 500 ppm of NH₃, 10% of O₂, and N₂ as a balance gas. The gas hourly space velocity (GHS/) was 80 000 h⁻¹ at temperature of the feed stream of 100 to 550° C. The results are illustrated in FIG. 7.

As can be seen from FIG. 7, from 175 to 350° C., a 100% NOx conversion is attained with the catalyst comprising the zeolitic material obtained from Example 1 and with the comparative catalyst. Further, at 400° C., the NOx conversion obtained with the catalyst comprising the zeolitic material obtained from Example 1 is still of 100% while the NOx conversion obtained with the comparative catalyst decreases. Thus, the catalyst comprising the mesoporous zeolitic material having a framework structure type AEI of Example 1 permits to maintain the NO conversion compared to a catalyst comprising a (non-mesoporous) zeolitic material having a framework structure AEI. Furthermore, the NOx conversion obtained with the catalyst comprising the zeolitic material obtained from Example 2 is of 100% from 200° C. to 350° C. Further, at temperatures higher than 400° C. to 550° C., this catalyst permits to obtain higher NOx conversions of from approximately 97% to 80%, respectively, compared to the NOx conversion obtained with the comparative catalyst which are from approximately 97% to approximately 65%. Thus, the catalyst comprising the mesoporous zeolitic material having a framework structure type AEI of

Example 2 according to the present invention permits to provide an improved NOx conversion at high temperatures compared to a catalyst comprising the zeolitic material having a framework structure type AEI of Comparative Example 1 not according to the present invention.

### Comparative Example 2

### Preparation of a Zeolitic Material having a Framework Type AEI not According to the Present Invention in the Absence of dimethylocta decyl[3-(trimethoxysilyl)propyl]ammonium chloride

a) Preparing a zeolitic material having a framework type AEI

Materials:

| | | |
|---|---|---|
| Zeolite Y powder having a SiO₂:Al₂O₃ molar | 1 | g |
| ratio of 21.6:1 | | |
| N,N-diethyl-cis-2,6-dimethylpiperidinium hydroxide solution | 9.4 | g |
| (0.24M in water) as obtained in Reference Example 2 | | |
| NaOH powder | 0.24 | g |

9.4 g of N,N-diethyl-cis-2,6-dimethylpiperidinium hydroxide solution (0.24 M) (the first structure directing agent, FOSDA) was added dropwise to 1 g of zeolite Y in a beaker and the mixture was stirred magnetically for 30 minutes at room temperature. 0.24 g of NaOH was added and the mixture was stirred magnetically for 30 minutes. The synthesis mixture composition was 0.17 Na₂O: 0.14 FOSDA: 1.0 SiO₂: 0.05 Al₂O₃: 30 H₂O. The term SiO₂ refers to the silicon comprised in the zeolite Y calculated as silica. The obtained mixture was transferred in a Teflon-lined autoclave. The autoclave was sealed and the mixture crystallized at 140° C. for 3 days (72 hours) under rotation (at a constant speed of 50 rpm). After pressure release and cooling to room temperature, the obtained suspension was subjected to filtration. The filter cake was washed with deionized water and was then dried for 2 hours at a temperature of 100° C. 0.8 g of a zeolitic material was obtained. The SiO₂: Al₂O₃ molar ratio of the zeolitic material was 16.4.

The XRD patterns of the respectively obtained zeolitic material show the characteristic peaks of AEI framework structure, as shown in FIG. 8(a).

The SEM images of the respectively obtained AEI zeolitic material (magnification: scale bar 1 micrometer) are shown in FIG. 9 (a).

Elemental analysis of the respectively obtained zeolitic material determined as described in Reference Example 1 f):

C %—11.8

N %—1.2

C/N—9.8

The BET specific surface area of the respectively obtained zeolitic material, after calcination in air at 550° C. for 4 hours, was 534 m²/g determined as described in Reference Example 1 b).

The mesopore volume was of 0.02 cm³/g as determined as described in Reference Example 1 b) and the micropore volume was of 0.24 cm³/g as determined as described in Reference Example 1 b). The ratio of mesopore volume to micropore volume was 0.08:1 and the ratio of mesopore volume to the total pore volume was 0.08:1.

b) Preparing the H-form of a zeolitic material having a framework type AEI

The dried zeolitic material obtained from a) is ion-exchanged with a 1M NH₄NO₃ solution at 80° C. for 2 hours and calcined in air at 550° C. for 4 hours. The procedure was repeated once.

### Example 5

### Preparation of a Mesoporous Zeolitic Material having a Framework Type AEI

a) Preparing a mesoporous zeolitic material having a framework type AEI

Materials:

| | | |
|---|---|---|
| Zeolite Y powder having a SiO₂:Al₂O₃ molar | 1 | g |
| ratio of 21.6:1 | | |
| N,N-diethyl-cis-2,6-dimethylpiperidinium hydroxide solution | 9.4 | g |
| (0.24M in water) as obtained in Reference Example 2 | | |
| NaOH powder | 0.24 | g |
| Dimethyloctadecyl[3-(trimethoxysilyl)propyl]ammonium | 0.10 | g |
| chloride (65% concentration in methanol) | | |
| Seed crystals AEI (SSZ-39 - not mesoporous) | 0.02 | g |

9.4 g of N,N-diethyl-cis-2,6-dimethylpiperidinium hydroxide solution (0.24 M) (the first structure directing agent, FOSDA) was added dropwise to 1 g of zeolite Y in a beaker and the mixture was stirred magnetically for 30 minutes at room temperature. 0.24 g of NaOH was added and the mixture was stirred magnetically for 30 minutes.

0.10 g of dimethyloctadecyl[3-(trimethoxysilyl)propyl]ammonium chloride (the second structure directing agent, SOSDA) was added to the mixture and the resulting mixture was stirred magnetically for 4 hours at room temperature. Finally, 0.02 g of SSZ-39 seed crystals was added and the mixture was stirred for 10 minutes at room temperature. The synthesis mixture composition was 0.17 Na₂O: 0.14 FOSDA: 0.008 SOSDA: 1.0 SiO₂: 0.05 Al₂O₃: 30 H₂O. The term SiO₂ refers to the silicon comprised in the zeolite Y calculated as silica. The obtained mixture was transferred in a Teflon-lined autoclave. The autoclave was sealed and the mixture crystallized at 140° C. for 3 days (72 hours) under rotation (at a constant speed of 50 rpm). After pressure release and cooling to room temperature, the obtained suspension was subjected to filtration. The filter cake was washed with deionized water and was then dried for 2 hours at a temperature of 100° C. 0.8 g of a zeolitic material was obtained. The SiO₂: Al₂O₃ molar ratio of the zeolitic material was 16.2.

The XRD patterns of the respectively obtained zeolitic material show the characteristic peaks of AEI framework structure, as shown in FIG. 8(b).

The SEM images of the respectively obtained AEI zeolitic material (magnification: scale bar 1 micrometer) are shown in FIG. 9(b). It is noted that smaller crystals are observed compared to those of the zeolitic material of Comparative Example 2.

Elemental analysis of the respectively obtained zeolitic material determined as described in Reference Example 1 f):

C %—14.3

N %—1.3

C/N—11.0

The C/N ratio of this zeolitic material is higher than those of the zeolitic material of Comparative Example 2 (conventional SSZ-39).

The BET specific surface area of the respectively obtained zeolitic material, after calcination in air at 550° C. for 4 hours, was 559 m²/g determined as described in Reference Example 1 b).

The mesopore volume was of 0.17 cm³/g as determined as described in Reference Example 1 b) and the micropore volume was of 0.25 cm³/g as determined as described in Reference Example 1 b). The ratio of mesopore volume to micropore volume was 0.68:1 and the ratio of mesopore volume to the total pore volume was 0.4:1.

b) Preparing the H-form of a mesoporous zeolitic material having a framework type AEI

The dried zeolitic material obtained from a) is ion-exchanged with a 1M NH₄NO₃ solution at 80° C. for 2 hours and calcined in air at 550° C. for 4 hours. The procedure was repeated once.

### Example 6

### Preparation of a Mesoporous Zeolitic Material having a Framework Type AEI

a) Preparing a mesoporous zeolitic material having a framework type AEI

Materials:

| | | |
|---|---|---|
| Zeolite Y powder having a SiO₂:Al₂O₃ molar | 1 | g |
| ratio of 21.6:1 | | |
| N,N-diethyl-cis-2,6-dimethylpiperidinium hydroxide solution | 9.4 | g |
| (0.24M in water) as obtained in Reference Example 2 | | |
| NaOH powder | 0.24 | g |
| Dimethyloctadecyl[3-(trimethoxysilyl)propyl]ammonium | 0.20 | g |
| chloride (65% concentration in methanol) | | |
| Seed crystals AEI (SSZ-39 - not mesoporous) | 0.02 | g |

9.4 g of N,N-diethyl-cis-2,6-dimethylpiperidinium hydroxide solution (0.24 M) (the first structure directing agent, FOSDA) was added dropwise to 1 g of zeolite Y in a beaker and the mixture was stirred magnetically for 30 minutes at room temperature. 0.24 g of NaOH was added and the mixture was stirred magnetically for 30 minutes.

0.20 g of dimethyloctadecyl[3-(trimethoxysilyl)propyl]ammonium chloride (the second structure directing agent, SOSDA) was added to the mixture and the resulting mixture was stirred magnetically for 4 hours at room temperature. Finally, 0.02 g of SSZ-39 seed crystals was added and the mixture was stirred for 10 minutes at room temperature. The synthesis mixture composition was 0.17 Na₂O: 0.14 FOSDA: 0.016 SOSDA: 1.0 SiO₂: 0.05 Al₂O₃: 30 H₂O. The term SiO₂ refers to the silicon comprised in the zeolite Y calculated as silica. The obtained mixture was transferred in a Teflon-lined autoclave. The autoclave was sealed and the mixture crystallized at 140° C. for 3 days (72 hours) under rotation (at a constant speed of 50 rpm). After pressure release and cooling to room temperature, the obtained suspension was subjected to filtration. The filter cake was washed with deionized water and was then dried for 2 hours at a temperature of 100° C. 0.8 g of a zeolitic material (SSZ-39) was obtained. The SiO₂: Al₂O₃ molar ratio of the zeolitic material was 17.4.

The XRD patterns of the respectively obtained zeolitic material show the characteristic peaks of AEI framework structure, as shown in FIG. 8(c).

The SEM images of the respectively obtained fresh AEI zeolitic material (magnification: scale bar 1 micrometer) are shown in FIG. 9(c). The crystallinity of the sample was of approximately 100%, determined as described in Reference Example 1 e), as shown in FIG. 10.

Elemental analysis of the respectively obtained zeolitic material determined as described in Reference Example 1 f):

C %—16.8

N %—1.5

C/N—11.2

The C/N ratio of this zeolitic material is higher than those of the zeolitic material of Comparative Example (conventional SSZ-39) and those of the mesoporous zeolitic material AEI of Example 5. Thus, this shows that the C/N ratio increases with the amount of SOSDA. Thus, without wanting to be bound to any theory, it is believed that the SOSDA is encapsulated in the zeolitic materials.

The BET specific surface area of the respectively obtained zeolitic material, after calcination in air at 550° C. for 4 hours, was 566 m²/g determined as described in Reference Example 1 b).

The mesopore volume was of 0.28 cm³/g as determined as described in Reference Example 1 b) and the micropore volume was of 0.24 cm³/g as determined as described in Reference Example 1 b). The ratio of mesopore volume to micropore volume was 1.17:1 and the ratio of mesopore volume to the total pore volume was 0.54:1.

b) Preparing the H-form of a mesoporous zeolitic material having a framework type AEI

The dried zeolitic material obtained from a) is ion-exchanged with a 1M NH₄NO₃ solution at 80° C. for 2 hours and calcined in air at 550° C. for 4 hours. The procedure was repeated once.

### Comparative Example 3

### Attempts to Prepare Mesoporous Zeolitic Materials having a Framework Type AEI

The zeolitic materials of Comparative Examples 3a-3g were prepared as the zeolitic materials of Example 5 except that the conditions outlined in Table 1 below were applied.

TABLE 1
Conditions of the synthesis of the zeolitic
materials of Comparative Examples 3a-3d

| | | Na₂O:SiO₂ | SOSDA:SiO₂ | Seeds:SiO₂ | |
|---|---|---|---|---|---|
| | SiO₂:Al₂O₃ | in the | in the | in the | |
| Comp. | molar ratio | synthesis | synthesis | synthesis | |
| Example 3 | of zeolite Y | mixture | mixture | mixture | Products |
| a | 21.6:1 | 0.13:1 | 0.016:1 | 0.02:1 | FAU |
| b | 21.6:1 | 0.17:1 | 0.016:1 | 0 | FAU |
| c | 11:1 | 0.17:1 | 0.016:1 | 0.02:1 | FAU |
| d | 256:1 | 0.17:1 | 0.016:1 | 0.02:1 | FAU |

### Example 7

### Preparation of Mesoporous Zeolitic Materials having a Framework Type AEI

The zeolitic materials of Examples 7a-7c were prepared as the zeolitic materials of Example 5 except that the conditions outlined in Table 2 below were applied.

TABLE 2
Conditions of the synthesis of the zeolitic materials of Examples 7a-7c

| | | Na₂O:SiO₂ | SOSDA:SiO₂ | Seeds:SiO₂ | |
|---|---|---|---|---|---|
| | SiO₂:Al₂O₃ | in the | in the | in the | |
| | molar ratio | synthesis | synthesis | synthesis | |
| Example 7 | of zeolite Y | mixture | mixture | mixture | Products** |
| a | 21.6:1 | 0.17:1 | 0.024:1 | 0.02:1 | SSZ-39* + MOR |
| b | 21.6:1 | 0.20:1 | 0.016:1 | 0.02:1 | SSZ-39* + MOR |
| c | 35:1 | 0.17:1 | 0.016:1 | 0.02:1 | SSZ-39* + MOR + |
| | | | | | FAU |

*mesoporous SSZ-39
**MOR and FAU are impurities.

As may be taken from Tables 1 and 2, the ratios of SOSDA/SiO₂ and Na₂O/SiO₂, the SiO₂/Al₂O₃ ratio of the zeolite Y as starting materials, and the addition of SSZ-39 zeolite seeds are critical for the successful synthesis of mesoporous zeolitic material having a framework type AEI.

When the SOSDA/SiO₂ ratio was of 0.024, a mesoporous SSZ-39 zeolitic material was obtained with a zeolitic material MOR as impurity phase (7a). When the Na₂O/SiO₂ ratio was lower than 0.13, the product was the starting material zeolite Y (3a). Increasing the Na₂O/SiO₂ ratio to 0.20 permitted to obtain a mesoporous zeolitic material SSZ-39 with a zeolitic material MOR as impurity phase (7b). A Na₂O/SiO₂ ratio in the starting gel of about 0.17 permitted to obtain only a mesoporous zeolitic material SSZ-39 as may be taken from Examples 5 and 6. Moreover, when the starting gel is crystallized without addition of SSZ-39 seeds, the product was always a zeolitic material FAU (3b). On the contrary, when the SSZ-39 seeds were added, mesoporous SSZ-39 zeolitic materials with high crystallinity were obtained as illustrated by Examples 5 and 6. In addition, it is further believed that the SiO₂/Al₂O₃ molar ratio of the starting zeolite Y is of great importance in the synthesis of mesoporous AEI zeolitic materials. As may be taken from Tables 1 and 2, when the SiO₂/Al₂O₃ molar ratio of Y zeolite was 11.0, there was no interzeolite transformation and the product was the starting material (3c). Further, increasing the SiO₂/Al₂O₃ ratio of zeolite Y to 35.0, resulted in a mesoporous zeolitic, material SSZ-39 and the formation of impurities, a zeolitic material MOR mixed with zeolite Y, the starting material (7c). Finally, when the SiO₂/Al₂O₃ ratio of zeolite Y was further increased to 256, there was no transformation (3d). However, with a SiO₂/Al₂O₃ ratio of zeolite Y of 21.6, mesoporous SSZ-39 zeolitic materials with high crystallinity were obtained as illustrated by Examples 5 and 6.

### Example 8

### Use of Zeolitic Materials having a Framework Type AEI for Catalytically Converting Methanol to one or More Olefins (MTO)

Methanol-to-olefin (MTO) reactions were carried out in a fixed-bed reactor at atmospheric pressure. A total of 0.5 g of a catalyst comprising the zeolitic material according to b) of Example 6 (20-40 mesh—Catalyst b below) was loaded into the reactor. Said catalyst was prepared by tableting the H-form of the zeolitic material according to b) of Example 6 to 20-40 mesh. The sample was pretreated in flowing nitrogen at 500° C. for 2 hours, and then the temperature of the reactor was decreased to 350° C. CH₃OH was pumped into the reactor under nitrogen. The weight hourly space velocity (WHSV) was 0.8 h⁻¹. Same was performed for a catalyst comprising the zeolitic material according to b) of Comparative Example 2 (Catalyst a below). The products were analyzed online using an Agilent 6890 gas chromatograph equipped with a FID detector and an HP-PONA methyl siloxane capillary column.

The catalytic data in MTO reactions at a reaction e of 60 min at 350° C. are displayed in Table 3 below.

TABLE 3

| | Conversion | Selectivities (%) | | | |
|---|---|---|---|---|---|
| | (%) | C₂H₄ | C₃H₆ | C₄H₈ | C₁-C₄ alkanes |
| Catalyst a | 100 | 6.94 | 20.4 | 11.25 | 45.64 |
| (Comparative) | | | | | |
| Catalyst b | 100 | 9.57 | 21.05 | 13.52 | 45.03 |
| (Inventive) | | | | | |

The dependence of the methanol conversion on time in MTO is illustrated in FIG. 14. As may be taken from said figure, the catalyst b (comprising the mesoporous AEI zeolitic material) has a much longer lifetime compared to catalyst a. Indeed, the methanol conversion over the comparative catalyst a starts to decrease at 180 min while the methanol conversion over the inventive catalyst b is still at 100% at about 960 minutes. This longer lifetime is reasonably attributed to the presence of mesoporosity in the zeolitic material of Example 6 which is very favorable for fast mass transfer and coke resistance. In particular, as shown in FIG. 16, the coke weight loss of the inventive catalyst b (mesoporous) reacted for 960 minutes is much lower than that of the comparative catalyst a reacted for 780 minutes. Thus, this demonstrates that the mesoporous zeolitic material AEI improves the prevention of coke formation compared to a non-mesoporous zeolitic material AEI.

### BRIEF DESCRIPTION OF THE FIGURES

FIG. 1: shows the XRD patterns of the respectively obtained AEI zeolitic materials according to a) of Examples 1 and 2 and of Comparative Example 1

FIG. 2a: shows the SEM image of the respectively obtained fresh AEI zeolitic material (low magnification: scale bar 1 micrometer) according to a) of Example 1.

FIG. 2b: shows the SEM image of the respectively obtained fresh AEI zeolitic material (high magnification: scale bar 500 nanometers) according to a) of Example 1.

FIG. 3: shows the crystallization curve of the zeolitic material according to a) of Example 1.

FIG. 4: shows the XRD patterns of the respectively obtained AEI zeolitic material according to a) of Example 1 after crystallization duration of 0 hour to 11 days. After 6 hours of crystallization, the XRD pattern of the zeolitic material shows the characteristic peaks related to zeolite Y (starting material), namely a high intensity peak around 6° (2Theta), a high intensity peak around 12° (2Theta), a high intensity peak around 16° (2Theta), a high intensity peak around 24° (2Theta) and a high intensity peak around 27° (2Theta). Alter 3 days of crystallization, the XRD pattern of the zeolitic material shows peaks associated with the framework structure AEI, namely a peak at around 9.5° 2Theta (highest intensity), a peak at around 16.1° 2Theta, a peak at around 16.9° 2Theta, a peak at around 17.2° 2Theta, a peak at around 20.7° 2Theta, a peak at around 21.4° 2Theta, a peak at 24.0 2Theta, a peak at 26.3° 2Theta and a peak at 31.2° 2Theta. After 5-7 days of crystallization, the XRD pattern of the zeolitic material shows the characteristic peaks of the framework structure type AEI. Further, increasing the crystallization to 9 days and 11 days does not change the intensity of the peaks of the XRD patterns associated with the framework structure type AEI. This illustrates that the zeolitic, material having a framework structure type AEI obtained according to the invention has a high stability in the synthesis mixture.

FIG. 5a: shows the SEM image of the respectively obtained fresh AEI zeolitic material (low magnification: scale bar 1 micrometer) according to a) of Example 2.

FIG. 5b: shows the SEM image of the respectively obtained fresh AEI zeolitic material (high magnification: scale bar 500 nanometers) according to a) of Example 2.

FIG. 6a: shows the SEM image of the respectively obtained fresh AEI zeolitic material (low magnification: scale bar 1 micrometer) according to a) of Comparative Example 1.

FIG. 6b: shows the SEM image of the respectively obtained fresh AEI zeolitic material (high magnification: scale bar 500 nanometers) according to a) of Comparative Example 1.

FIG. 7: shows the NOx conversion of fresh catalysts comprising a zeolitic material according to Examples 1, 2 and Comparative Example 1.

FIG. 8: shows the XRD patterns of the respectively obtained AEI zeolitic materials according to a) of Examples 5 (c) arid 6 (b) and of Comparative Example 2 (a).

FIG. 9: shows the SEM images of the respectively obtained AEI zeolitic materials according to a) of Examples 5 (c) and 6 (b) and of Comparative Example 2 (a) (magnification: scale bar 1 micrometer).

FIG. 10: shows the crystallization curve of the zeolitic material according to a) of Example 6.

FIG. 11: shows N₂ sorption isotherms of the zeolitic materials of Examples 5 (b), 6 (c) and Comparative Example 2 (a) as determined in Reference Example 1 b) (FINESORB-3020M) herein. Obvious hysteresis loops in the range of 0.7<PIP₀<1.0 can be observed for the zeolitic material of Example 5 and the zeolitic material of Example 6 indicating the existence of mesoporosity in the samples.

FIG. 12: shows the XRD patterns of the respectively obtained AEI zeolitic material according to a) of Example 6 after crystallization duration of 0 hour to 268 hours. After 9 hours of crystallization, the XRD pattern of the zeolitic material shows weak peaks related to AEI zeolitic material. Increasing the crystallization from 12 to 48 hours, the XRD peaks associated with AEI zeolitic material gradually increase, together with the gradual decrease of the characteristic peaks related to the starting material (zeolite Y). When the crystallization reaches 72 hours, the XRD pattern of the zeolitic material shows the characteristic peaks of the framework structure type AEI. Further, increasing the crystallization to 264 hours does not change the intensity of the peaks of the XRD patterns associated with the framework structure type AEI.

FIG. 13: shows the NH₃-TPD curves of the zeolitic materials according to b) of Comparative Example 2 (a) and to b) of Example 6 (b). The NH₃-TPD curves are determined as in Reference Example 1 g) herein. Both samples have similar peak position for ammonia desorption, giving at about 170° C. and about 510° C. Notably, the peak intensity of the H-SSZ-39 (a) is stronger than that of the mesoporous H-SSZ-39 (b), which is attributed to more four-coordinated Al species in the H-SSZ-39 than those in the mesoporous H-SSZ-39.

FIG. 14: shows the methanol conversion in MTO reaction using the zeoltic materials of Comparative Example 2 (a) and of Example 6 (b) on a stream at 350° C.

FIG. 15: shows the selectivities in MTO reaction for ethylene (a), propylene (b), butane (c) and C₁-C₄ alkane (d) over the zeolitic materials of Comparative Example 2 (A) and of Example 6 (B).

FIG. 16: shows the TG-DTA curves of the comparative catalyst a after reaction during 780 minutes (a) and of the inventive catalyst b (mesoporous) after reaction during 960 minutes. The TG-DTA curves are determined as in Reference Example 1 h) herein.

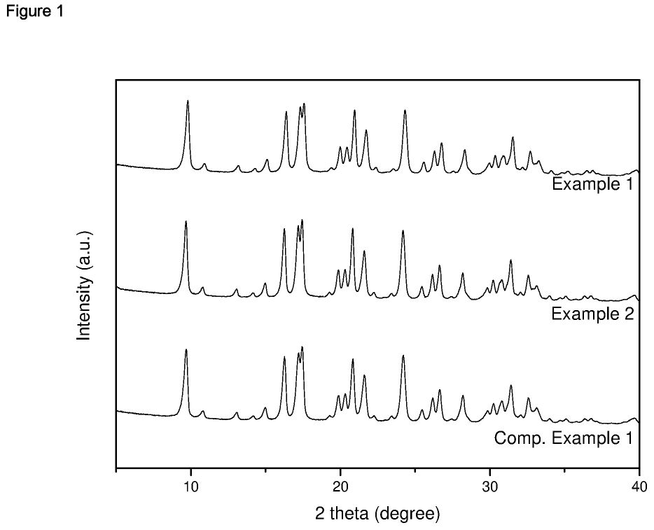

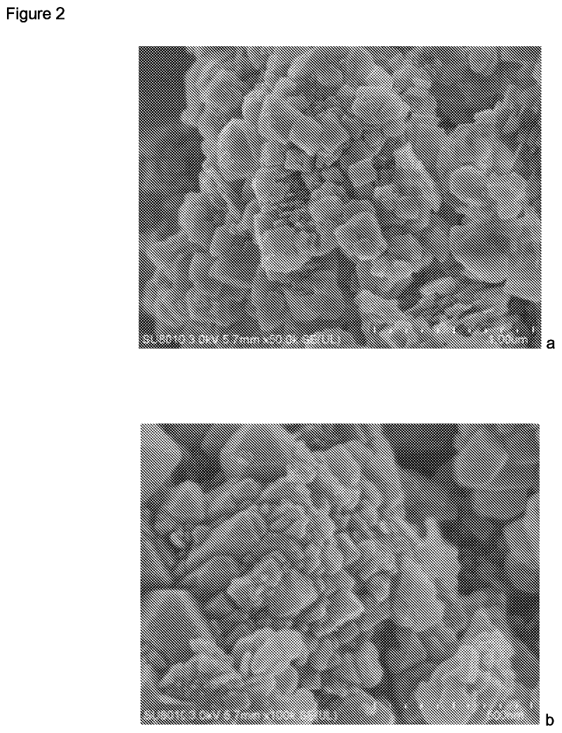

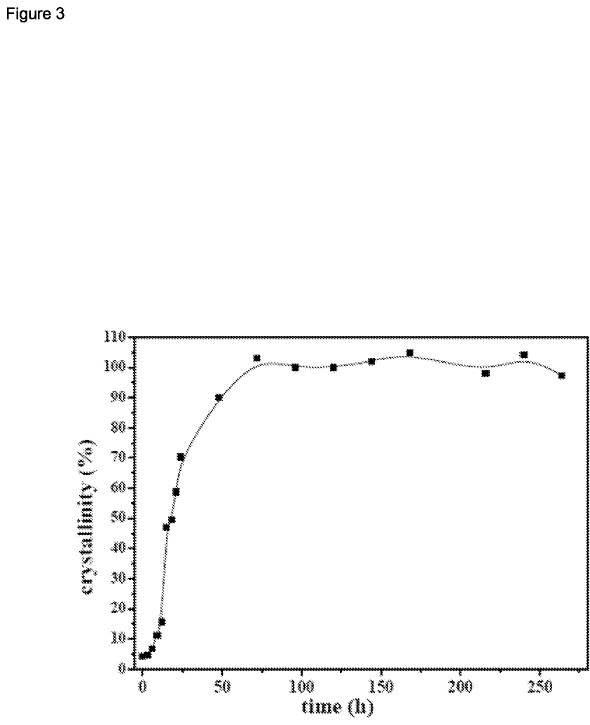

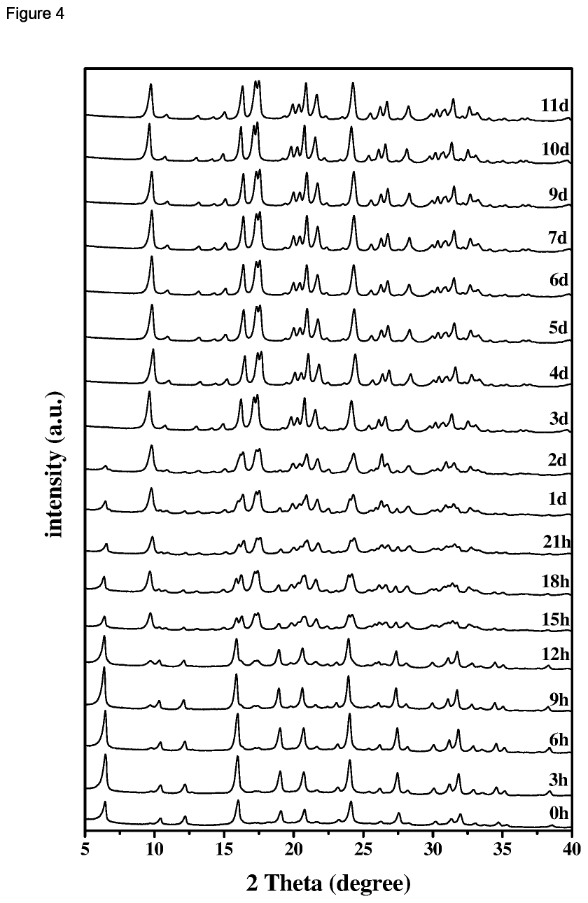

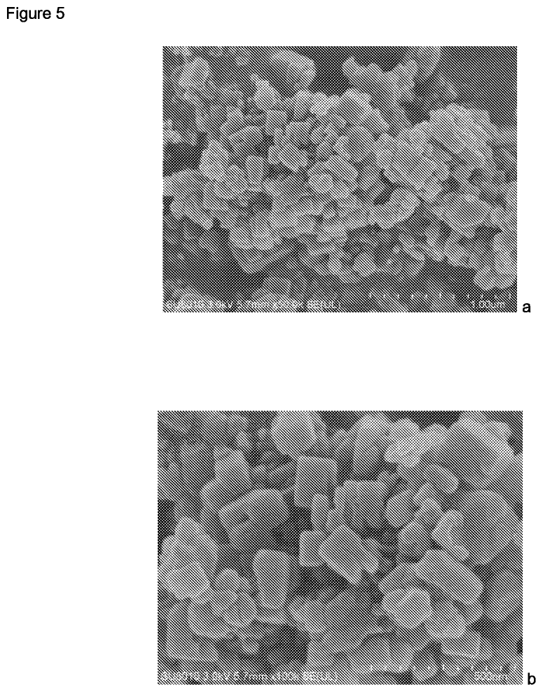

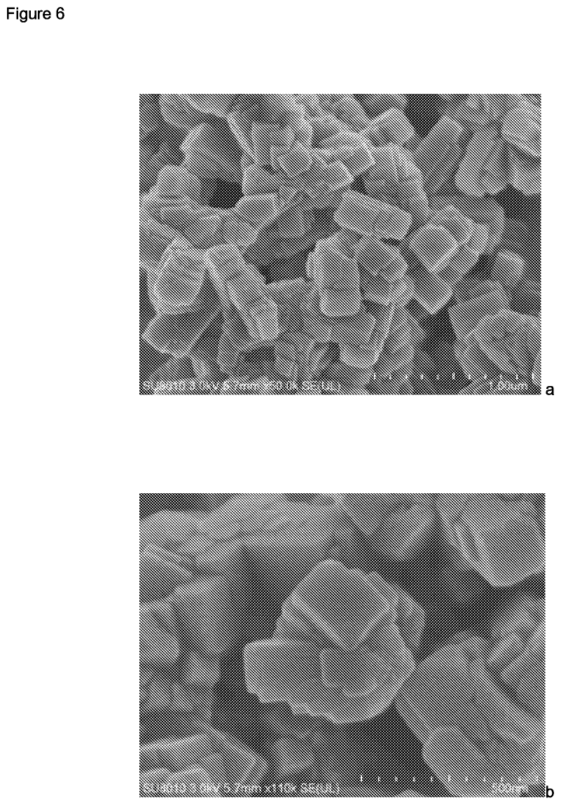

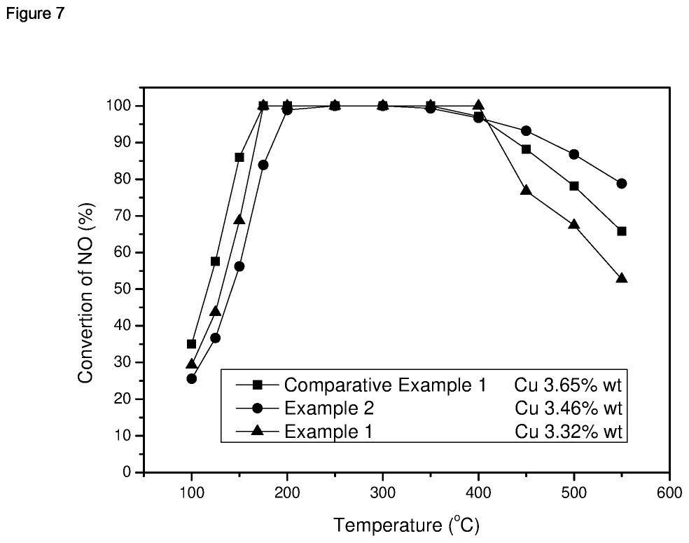

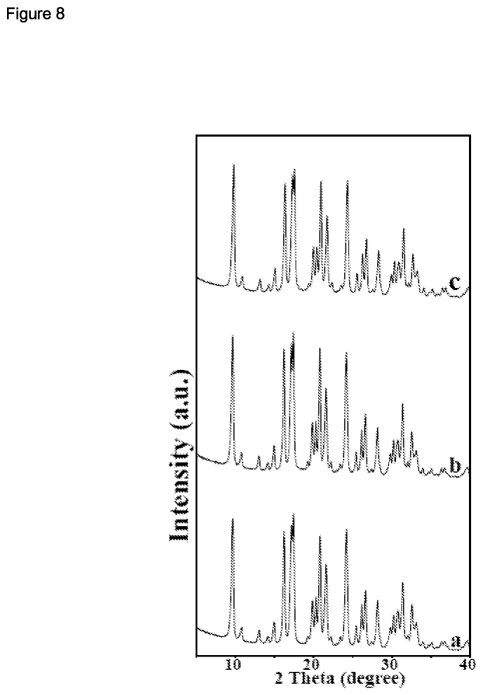

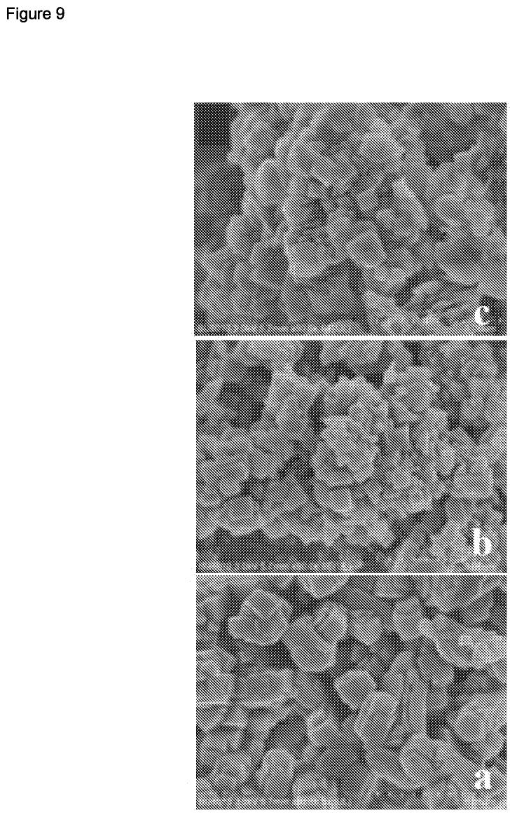

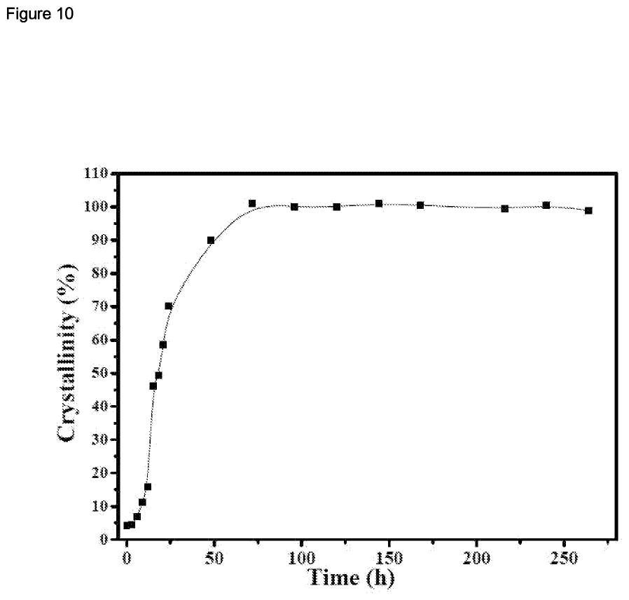

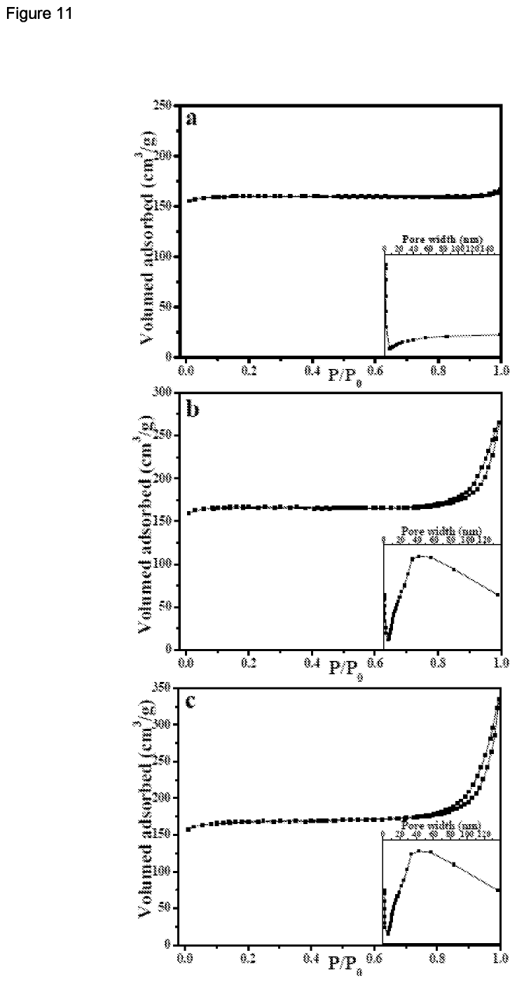

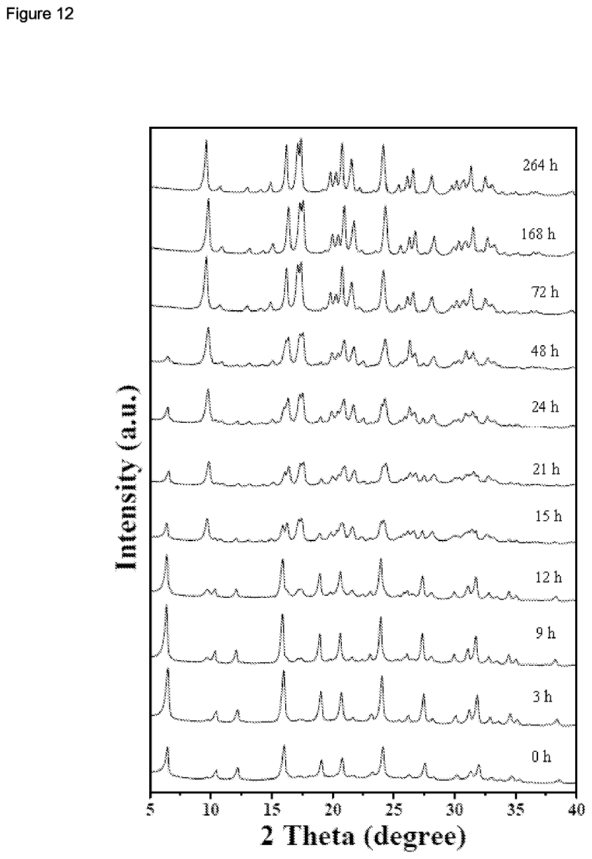

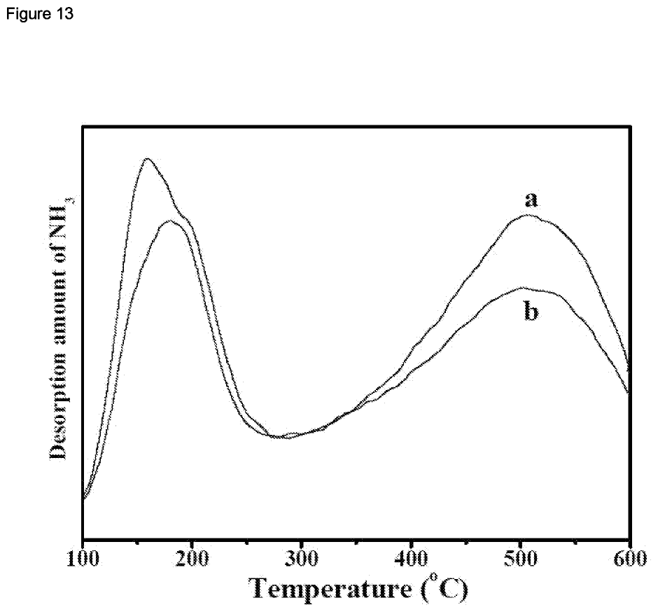

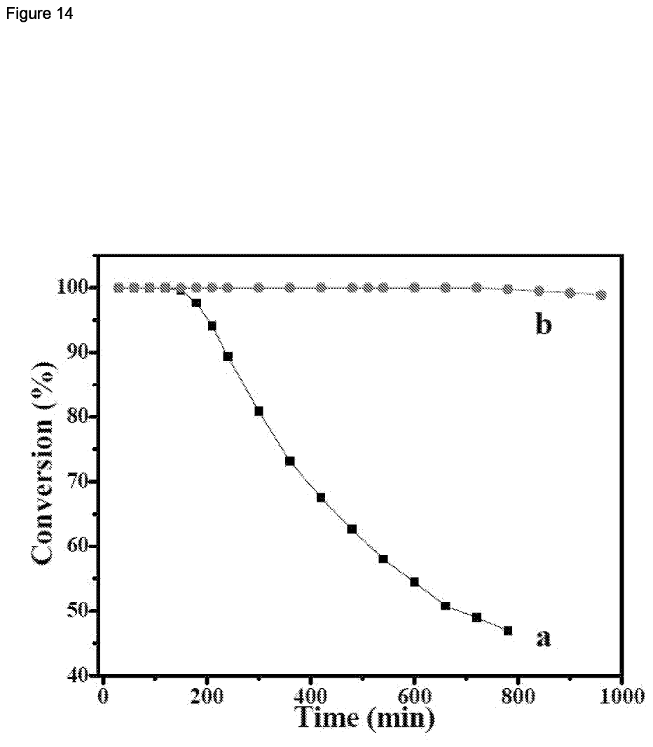

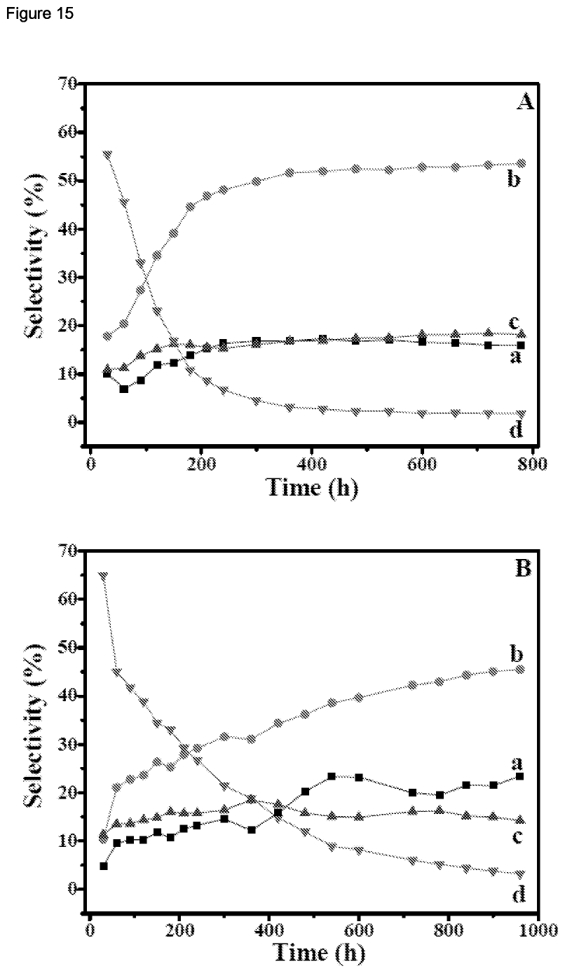

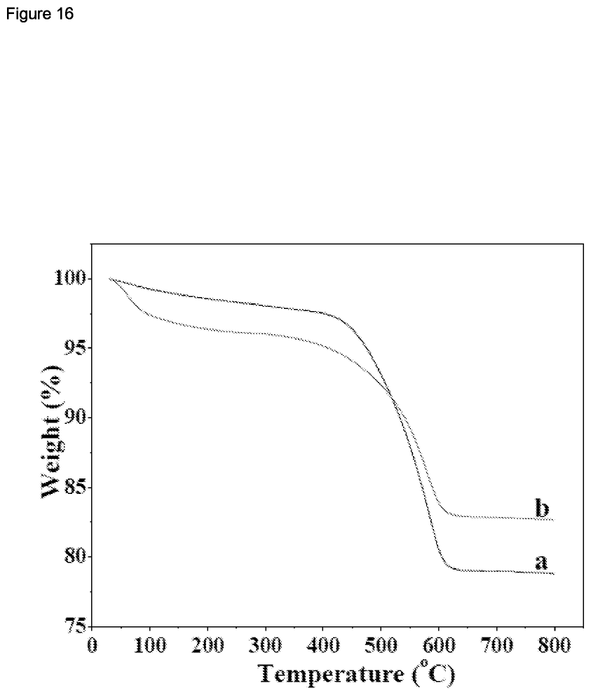

### CITED LITERATURE

CN 107285333 A

CN 107285334 A

1. A process for preparing a porous oxidic material the process comprising:
crystallizing, at a crystallization temperature in the range of from 90 to 200° C., a synthesis mixture, to obtain a mother liquor comprising the porous oxidic material comprising said zeolitic material having an AEI framework,
wherein the synthesis mixture comprises a zeolitic material having an FAU framework comprising a tetravalent element Y, a trivalent element X, and O, and water, a base source, a first organic structure directing agent as an AEI framework structure directing agent, a second organic structure directing agent comprising a dimethyl-octadecyl[3-(trimethoxysilyl)-propyl]ammonium cation, and seed crystals,
wherein Y comprises Si, Sn, Ti, Zr, and/or Ge,
wherein X comprises Al, B, In, and/or Ga, and
wherein the porous oxidic material comprises micropores and mesopores, and a zeolitic material having an AEI framework comprising a tetravalent element, Y, a trivalent element, X, and oxygen, the micropores having a pore diameter determined by nitrogen adsorption-desorption at 77 K of less than 2 nm and the mesopores having a pore diameter determined by nitrogen adsorption-desorption at 77 K in a range of from 2 to 50 nm.

2. The process of claim 1, wherein the first structure directing agent comprises:
a quaternary phosphonium cation comprising compound; and
a N,N-diethyl-2,6-dimethylpiperidinium cation comprising compound.

3. The process of claim 1, wherein the second organic structure directing agent comprises a salt of the dimethyloctadecyl[3-(trimethoxysilyl)propyl]-ammonium cation.

4. The process of claim 1, wherein Y is Si.

5. The process of claim 1, wherein the zeolitic material having the FAU framework type a faujasite zeolite, a zeolite Y, a zeolite X, an LSZ-210 zeolite, and/or a zeolite USY, and
wherein, in the FAU framework, a molar ratio of Y:X, calculated as YO₂:X₂O₃, is optionally in a range of from 5:1 to 100:1.

6. The process of claim 1, wherein, in the synthesis mixture, a molar ratio of the first organic structure directing agent, FOSDA, relative to Y, calculated as FOSDA:YO₂, is in a range of from 0.05:1 to 0.30:1.

7. The process of claim 1, wherein, in the synthesis mixture, a molar ratio of the second organic structure directing agent, SOSDA, relative to Y, calculated as SOSDA:YO₂, is in a range of from 0.001:1 to 0.070:1.

8. The process of claim 1, wherein, in the synthesis mixture, a molar ratio of the base source of a base relative to Y, calculated as base source: YO₂, is in a range of from 0.10:1 to 0.70:1.

9. The process of claim 1, wherein, in the synthesis mixture, a molar ratio of H₂O relative to Y, calculated as H₂O:YO₂, is in a range of from 2:1 to 80:1.

10. The process of claim 1, wherein, the seed crystals comprise a zeolitic material having an AEI, CHA, or RTH framework.
wherein, in the synthesis mixture, a weight ratio of the seed crystals to the zeolitic material having the FAU framework is optionally in a range of from 0.001:1 to 0.1:1.

11. The process of claim 1, wherein the synthesis mixture is prepared by a process comprising:
(i.1) preparing a first mixture comprising the zeolitic material having the FAU framework comprising the tetravalent element Y, trivalent element X, oxygen, water, and the first organic structure directing agent;
(i.2) adding the base source to the first mixture to obtain a second mixture;
(i.3) adding the second organic structure directing agent to the second mixture to obtain a third mixture;
(i.4) adding the seed crystals to the third mixture, to obtain the synthesis mixture.

12. The process of claim 1, wherein the hydrothermally crystallizing comprises a crystallization duration in a range of from 0.75 to 20 days.

13. The process of claim 1, wherein during hydrothermally crystallizing the synthesis mixture is agitated.

14. The process of claim 1, further comprising:
(iii) optionally cooling the mother liquor comprising the porous oxidic material comprising the zeolitic material having the AEl framework.
(iv) separating the porous oxidic material from the mother liquor; and
(vi) optionally subjecting the porous oxidic material after (iv) to ion-exchange conditions.

15. The process of claim 1, wherein the micropores have a micropore volume and the mesopores have a mesopore volume,
wherein a ratio of the mesopore volume to the micropore volume of the porous oxidic material is at least 0.5:1, and
wherein a ratio of the mesopore volume to a total pore volume of the porous oxidic material is at least 0.3:1.

16. The process of claim 14, wherein the subjecting (vi) comprises:
(vi.1) bringing a solution comprising ammonium ions into contact with the porous oxidic material, to obtain a porous oxidic material in its ammonium form;
(vi.2) calcining the porous oxidic material obtained in (vi.1) in a gas atmosphere, to obtain the H-form of the porous oxidic material;
(vi.3) optionally bringing a solution comprising transition metal ion into contact with the porous oxidic material obtained from (vi.2) under ion-exchange conditions;
(vi.4) calcining the porous oxidic material obtained in (vi.3) or after the calcining (vi.2) in a gas atmosphere.

17. A porous oxidic material, comprising: micropores;
mesopores:
a zeolitic material having an AEI framework comprising a tetravalent element Y, a trivalent element X, and oxygen,
wherein the micropores have a pore diameter, determined by nitrogen adsorption-desorption at 77 K, of less than 2 nm,
wherein the mesopores have a pore diameter, determined by nitrogen adsorption-desorption at 77 K, in a range of from 2 to 50 nm,
wherein Y is Si, Sn, Ti, Zr, and/or Ge,
wherein X is Al, B, In, and/or Ga,
wherein the micropores have a micropore volume and the mesopores have a mesopore volume,
wherein a ratio of the mesopore volume to the micropore volume is at least 0.5:1, and
wherein a ratio of the mesopore volume to a total pore volume of the porous oxidic material is at least 0.3:1.

18. The material of claim 17, wherein, in the AEI framework, a molar ratio of Y:X, calculated as a YO₂:X₂O₃, is in a range of from 2:1 to 40:1.

19. The material of claim 17, having a BET specific surface area, determined N₂ sorption isotherms at liquid nitrogen temperature using a MICROMERITICS ASAP 2020M or FINESORB-3020M instrument and a TRISTAR system in a range of from 500 to 900 m²/g.

20. The material of claim 17, wherein the mesopore volume is in the range of from 0.15 to 0.80 cm³/g.

21. The porous oxidic material of claim 17, wherein the ratio of the mesopore volume to the micropore volume is in the range of from 0.5:1 to 3:1.

22. The material of claim 17, wherein the zeolitic material having the AIE framework has an X-ray diffraction pattern which comprising, with CuK (α1), reflections at:
a diffraction angle 2 θ of 8.5 to 10.5°, and an intensity of 90 to 100%;
a diffraction angle 2 θ of 15.1 to 17.1°, and an intensity of 75 to 95%;
a diffraction angle 2 θ of 15.9 to 17.9°, and an intensity of 80 to 100%;
a diffraction angle 2 θ of 16.2 to 18.2°, and an intensity of 80 to 100%;
a diffraction angle 2 θ of 19.7 to 21.7°, and an intensity of 80 to 100%;
a diffraction angle 2 θ of 20.4 to 22.4°, and an intensity of 50 to 70%;
a diffraction angle 2 θ of 23.2 to 25.2°, and an intensity of 80 to 100%;
a diffraction angle 2 θ of 25.3 to 27.3°, and an intensity of 30 to 50%;
a diffraction angle 2 θ of 30.2 to 32.2°, and an intensity of 40 to 60%;
wherein 100% relates to an intensity of a maximum peak in an X-ray powder diffraction pattern.

23. The material of claim 17, further comprising:
a transition metal.

24. A method for catalytically converting methanol to one or more olefins, the method comprising:
contacting a gas stream comprising methanol with a catalyst comprising the material of claim 17 in a reactor, obtaining a reaction mixture comprising one or more olefins.

25. A catalytically active material, as a catalyst, or as a catalyst component, comprising the material of claim 17.
